SEQUENCE LISTING (38 sequences)

SEQ ID NO: 1 - protein, 945 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 2 - protein, 315 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 3 - protein, 24 aa, Artificial Sequence
  Description of Artificial Sequence Synthetic peptide
DYKDDDDKDYKDDDDKDYKDDDDK

SEQ ID NO: 4 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 5 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 6 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 7 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 8 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 9 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 10 - protein, 171 aa, Nephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 11 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 12 - protein, 420 aa, Nephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 13 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 14 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 15 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 16 - protein, 32 aa, Euprosthenops australis
GGQGGQGQGRYGQGAGSSAAAAAAAAAAAAAA

SEQ ID NO: 17 - protein, 42 aa, Tetragnatha kauaiensis
GGLGGGQGAGQGGQQGAGQGGYGSGLGGAGQGASAAAAAAAA

SEQ ID NO: 18 - protein, 42 aa, Argiope aurantia
GGYGPGAGQQGPGSQGPGSGGQQGPGGLGPYGPSAAAAAAAA

SEQ ID NO: 19 - protein, 46 aa, Deinopis spinosa
GPGGYGGPGQQGPGQGQYGPGTGQQGQGPSGQQGPAGAAAAAAAAA

SEQ ID NO: 20 - protein, 42 aa, Nephila clavata
GPGGYGLGQQGPGQQGPGQQGPAGYGPSGLSGPGGAAAAAAA

SEQ ID NO: 21 - protein, 174 aa, Deinopis spinosa
GAGYGAGAGAGGGAGAGTGYGGGAGYGTGSGAGYGAGVGYGAGAGAGGGAGAGAGGGTGAGAGGGAGAGYGAGTGYGAGAGAGGGAGAGAGAGAGAGAGAGSGAGAGYGAGAGYGAGAGAGGVAGAGAAGGAGAAGGAGAAGGAGAAGGAGAGAGAGSGAGAGAGGGARAGAGG

SEQ ID NO: 22 - protein, 149 aa, Latrodectus hesperus
GGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 23 - protein, 161 aa, Nephila clavipes
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 24 - protein, 186 aa, Nephilengys cruentata
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 25 - protein, 132 aa, Uloborus diversus
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAA

SEQ ID NO: 26 - protein, 198 aa, Uloborus diversus
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 27 - protein, 190 aa, Araneus ventricosus
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 28 - protein, 166 aa, Dolomedes tenebrosus
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 29 - protein, 177 aa, Nephilengys cruentata
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 30 - protein, 174 aa, Nephilengys cruentata
GGAGQGGYGGLGGQGAGAAAAAAGGAGQGGYGGQGAGQGAAAAAASGAGQGGYGGPGAGQGAGAAAAAAGGAGQGGYGGLGGQGAGQGAGAAAAAAGGAGQGGYGGQGAGQGAAAAAAGGAGQGGYGGLGSGQGGYGGQGAGAAAAAGGAGQGGYGGLGGQGAGQGAGAAAAAA

SEQ ID NO: 31 - protein, 5 aa, Artificial Sequence
  Description of Artificial Sequence Synthetic peptide
SGAGG

SEQ ID NO: 32 - protein, 5 aa, Artificial Sequence
  Description of Artificial Sequence Synthetic peptide
GSGAG

SEQ ID NO: 33 - protein, 5 aa, Artificial Sequence
  Description of Artificial Sequence Synthetic peptide
GGSGA

SEQ ID NO: 34 - protein, 1600 aa, Artificial Sequence
  Description of Artificial Sequence Synthetic polypeptide
  MISC_FEATURE (7)..(11): This region may encompass “SGGQQ,” “GAGQQ,” “GQGPY,” “AGQQ” or “SQ,” wherein some positions may be absent
  MISC_FEATURE (15)..(19): This region may encompass “SGGQQ,” “GAGQQ,” “GQGPY,” “AGQQ” or “SQ,” wherein some positions may be absent
  MISC_FEATURE (23)..(27): This region may encompass “SGGQQ,” “GAGQQ,” “GQGPY,” “AGQQ” or “SQ,” wherein some positions may be absent
  MISC_FEATURE (31)..(35): This region may encompass “SGGQQ,” “GAGQQ,” “GQGPY,” “AGQQ” or “SQ,” wherein some positions may be absent
  MISC_FEATURE (39)..(43): This region may encompass “SGGQQ,” “GAGQQ,” “GQGPY,” “AGQQ” or “SQ,” wherein some positions may be absent
  MISC_FEATURE (47)..(51): This region may encompass “SGGQQ,” “GAGQQ,” “GQGPY,” “AGQQ” or “SQ,” wherein some positions may be absent
  MISC_FEATURE (55)..(59): This region may encompass “SGGQQ,” “GAGQQ,” “GQGPY,” “AGQQ” or “SQ,” wherein some positions may be absent
  MISC_FEATURE (63)..(67): This region may encompass “SGGQQ,” “GAGQQ,” “GQGPY,” “AGQQ” or “SQ,” wherein some positions may be absent
  MISC_FEATURE (4)..(67): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (71)..(80): This region may encompass 6-10 residues
  MISC_FEATURE (87)..(91): This region may encompass “SGGQQ,” “GAGQQ,” “GQGPY,” “AGQQ” or “SQ,” wherein some positions may be absent
  MISC_FEATURE (95)..(99): This region may encompass “SGGQQ,” “GAGQQ,” “GQGPY,” “AGQQ” or “SQ,” wherein some positions may be absent
  MISC_FEATURE (103)..(107): This region may encompass “SGGQQ,” “GAGQQ,” “GQGPY,” “AGQQ” or “SQ,” wherein some positions may be absent
  MISC_FEATURE (111)..(115): This region may encompass “SGGQQ,” “GAGQQ,” “GQGPY,” “AGQQ” or “SQ,” wherein some positions may be absent
  MISC_FEATURE (119)..(123): This region may encompass “SGGQQ,” “GAGQQ,” “GQGPY,” “AGQQ” or “SQ,” wherein some positions may be absent
  MISC_FEATURE (127)..(131): This region may encompass “SGGQQ,” “GAGQQ,” “GQGPY,” “AGQQ” or “SQ,” wherein some positions may be absent
  MISC_FEATURE (135)..(139): This region may encompass “SGGQQ,” “GAGQQ,” “GQGPY,” “AGQQ” or “SQ,” wherein some positions may be absent
  MISC_FEATURE (143)..(147): This region may encompass “SGGQQ,” “GAGQQ,” “GQGPY,” “AGQQ” or “SQ,” wherein some positions may be absent
  MISC_FEATURE (84)..(147): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (151)..(160): This region may encompass 6-10 residues
  MISC_FEATURE (167)..(171): This region may encompass “SGGQQ,” “GAGQQ,” “GQGPY,” “AGQQ” or “SQ,” wherein some positions may be absent
  MISC_FEATURE (175)..(179): This region may encompass “SGGQQ,” “GAGQQ,” “GQGPY,” “AGQQ” or “SQ,” wherein some positions may be absent
  MISC_FEATURE (183)..(187): This region may encompass “SGGQQ,” “GAGQQ,” “GQGPY,” “AGQQ” or “SQ,” wherein some positions may be absent
  MISC_FEATURE (191)..(195): This region may encompass “SGGQQ,” “GAGQQ,” “GQGPY,” “AGQQ” or “SQ,” wherein some positions may be absent
  MISC_FEATURE (199)..(203): This region may encompass “SGGQQ,” “GAGQQ,” “GQGPY,” “AGQQ” or “SQ,” wherein some positions may be absent
  MISC_FEATURE (207)..(211): This region may encompass “SGGQQ,” “GAGQQ,” “GQGPY,” “AGQQ” or “SQ,” wherein some positions may be absent
  MISC_FEATURE (215)..(219): This region may encompass “SGGQQ,” “GAGQQ,” “GQGPY,” “AGQQ” or “SQ,” wherein some positions may be absent
  MISC_FEATURE (223)..(227): This region may encompass “SGGQQ,” “GAGQQ,” “GQGPY,” “AGQQ” or “SQ,” wherein some positions may be absent
  MISC_FEATURE (164)..(227): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (231)..(240): This region may encompass 6-10 residues
  MISC_FEATURE (247)..(251): This region may encompass “SGGQQ,” “GAGQQ,” “GQGPY,” “AGQQ” or “SQ,” wherein some positions may be absent
  MISC_FEATURE (255)..(259): This region may encompass “SGGQQ,” “GAGQQ,” “GQGPY,” “AGQQ” or “SQ,” wherein some positions may be absent
  MISC_FEATURE (263)..(267): This region may encompass “SGGQQ,” “GAGQQ,” “GQGPY,” “AGQQ” or “SQ,” wherein some positions may be absent
  MISC_FEATURE (271)..(275): This region may encompass “SGGQQ,” “GAGQQ,” “GQGPY,” “AGQQ” or “SQ,” wherein some positions may be absent
  MISC_FEATURE (279)..(283): This region may encompass “SGGQQ,” “GAGQQ,” “GQGPY,” “AGQQ” or “SQ,” wherein some positions may be absent
  MISC_FEATURE (287)..(291): This region may encompass “SGGQQ,” “GAGQQ,” “GQGPY,” “AGQQ” or “SQ,” wherein some positions may be absent
  MISC_FEATURE (295)..(299): This region may encompass “SGGQQ,” “GAGQQ,” “GQGPY,” “AGQQ” or “SQ,” wherein some positions may be absent
  MISC_FEATURE (303)..(307): This region may encompass “SGGQQ,” “GAGQQ,” “GQGPY,” “AGQQ” or “SQ,” wherein some positions may be absent
  MISC_FEATURE (244)..(307): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (311)..(320): This region may encompass 6-10 residues
  MISC_FEATURE (327)..(331): This region may encompass “SGGQQ,” “GAGQQ,” “GQGPY,” “AGQQ” or “SQ,” wherein some positions may be absent
  MISC_FEATURE (335)..(339): This region may encompass “SGGQQ,” “GAGQQ,” “GQGPY,” “AGQQ” or “SQ,” wherein some positions may be absent
  MISC_FEATURE (343)..(347): This region may encompass “SGGQQ,” “GAGQQ,” “GQGPY,” “AGQQ” or “SQ,” wherein some positions may be absent
  MISC_FEATURE (351)..(355): This region may encompass “SGGQQ,” “GAGQQ,” “GQGPY,” “AGQQ” or “SQ,” wherein some positions may be absent
  MISC_FEATURE (359)..(363): This region may encompass “SGGQQ,” “GAGQQ,” “GQGPY,” “AGQQ” or “SQ,” wherein some positions may be absent
  MISC_FEATURE (367)..(371): This region may encompass “SGGQQ,” “GAGQQ,” “GQGPY,” “AGQQ” or “SQ,” wherein some positions may be absent
  MISC_FEATURE (375)..(379): This region may encompass “SGGQQ,” “GAGQQ,” “GQGPY,” “AGQQ” or “SQ,” wherein some positions may be absent
  MISC_FEATURE (383)..(387): This region may encompass “SGGQQ,” “GAGQQ,” “GQGPY,” “AGQQ” or “SQ,” wherein some positions may be absent
  MISC_FEATURE (324)..(387): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (391)..(400): This region may encompass 6-10 residues
  MISC_FEATURE (407)..(411): This region may encompass “SGGQQ,” “GAGQQ,” “GQGPY,” “AGQQ” or “SQ,” wherein some positions may be absent
  MISC_FEATURE (415)..(419): This region may encompass “SGGQQ,” “GAGQQ,” “GQGPY,” “AGQQ” or “SQ,” wherein some positions may be absent
  MISC_FEATURE (423)..(427): This region may encompass “SGGQQ,” “GAGQQ,” “GQGPY,” “AGQQ” or “SQ,” wherein some positions may be absent
  MISC_FEATURE (431)..(435): This region may encompass “SGGQQ,” “GAGQQ,” “GQGPY,” “AGQQ” or “SQ,” wherein some positions may be absent
  MISC_FEATURE (439)..(443): This region may encompass “SGGQQ,” “GAGQQ,” “GQGPY,” “AGQQ” or “SQ,” wherein some positions may be absent
  MISC_FEATURE (447)..(451): This region may encompass “SGGQQ,” “GAGQQ,” “GQGPY,” “AGQQ” or “SQ,” wherein some positions may be absent
  MISC_FEATURE (455)..(459): This region may encompass “SGGQQ,” “GAGQQ,” “GQGPY,” “AGQQ” or “SQ,” wherein some positions may be absent
  MISC_FEATURE (463)..(467): This region may encompass “SGGQQ,” “GAGQQ,” “GQGPY,” “AGQQ” or “SQ,” wherein some positions may be absent
  MISC_FEATURE (404)..(467): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (471)..(480): This region may encompass 6-10 residues
  MISC_FEATURE (487)..(491): This region may encompass “SGGQQ,” “GAGQQ,” “GQGPY,” “AGQQ” or “SQ,” wherein some positions may be absent
  MISC_FEATURE (495)..(499): This region may encompass “SGGQQ,” “GAGQQ,” “GQGPY,” “AGQQ” or “SQ,” wherein some positions may be absent
  MISC_FEATURE (503)..(507): This region may encompass “SGGQQ,” “GAGQQ,” “GQGPY,” “AGQQ” or “SQ,” wherein some positions may be absent
  MISC_FEATURE (511)..(515): This region may encompass “SGGQQ,” “GAGQQ,” “GQGPY,” “AGQQ” or “SQ,” wherein some positions may be absent
  MISC_FEATURE (519)..(523): This region may encompass “SGGQQ,” “GAGQQ,” “GQGPY,” “AGQQ” or “SQ,” wherein some positions may be absent
  MISC_FEATURE (527)..(531): This region may encompass “SGGQQ,” “GAGQQ,” “GQGPY,” “AGQQ” or “SQ,” wherein some positions may be absent
  MISC_FEATURE (535)..(539): This region may encompass “SGGQQ,” “GAGQQ,” “GQGPY,” “AGQQ” or “SQ,” wherein some positions may be absent
  MISC_FEATURE (543)..(547): This region may encompass “SGGQQ,” “GAGQQ,” “GQGPY,” “AGQQ” or “SQ,” wherein some positions may be absent
  MISC_FEATURE (484)..(547): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (551)..(560): This region may encompass 6-10 residues
  MISC_FEATURE (567)..(571): This region may encompass “SGGQQ,” “GAGQQ,” “GQGPY,” “AGQQ” or “SQ,” wherein some positions may be absent
  MISC_FEATURE (575)..(579): This region may encompass “SGGQQ,” “GAGQQ,” “GQGPY,” “AGQQ” or “SQ,” wherein some positions may be absent
  MISC_FEATURE (583)..(587): This region may encompass “SGGQQ,” “GAGQQ,” “GQGPY,” “AGQQ” or “SQ,” wherein some positions may be absent
  MISC_FEATURE (591)..(595): This region may encompass “SGGQQ,” “GAGQQ,” “GQGPY,” “AGQQ” or “SQ,” wherein some positions may be absent
  MISC_FEATURE (599)..(603): This region may encompass “SGGQQ,” “GAGQQ,” “GQGPY,” “AGQQ” or “SQ,” wherein some positions may be absent
  MISC_FEATURE (607)..(611): This region may encompass “SGGQQ,” “GAGQQ,” “GQGPY,” “AGQQ” or “SQ,” wherein some positions may be absent
  MISC_FEATURE (615)..(619): This region may encompass “SGGQQ,” “GAGQQ,” “GQGPY,” “AGQQ” or “SQ,” wherein some positions may be absent
  MISC_FEATURE (623)..(627): This region may encompass “SGGQQ,” “GAGQQ,” “GQGPY,” “AGQQ” or “SQ,” wherein some positions may be absent
  MISC_FEATURE (564)..(627): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (631)..(640): This region may encompass 6-10 residues
  MISC_FEATURE (647)..(651): This region may encompass “SGGQQ,” “GAGQQ,” “GQGPY,” “AGQQ” or “SQ,” wherein some positions may be absent
  MISC_FEATURE (655)..(659): This region may encompass “SGGQQ,” “GAGQQ,” “GQGPY,” “AGQQ” or “SQ,” wherein some positions may be absent
  MISC_FEATURE (663)..(667): This region may encompass “SGGQQ,” “GAGQQ,” “GQGPY,” “AGQQ” or “SQ,” wherein some positions may be absent
  MISC_FEATURE (671)..(675): This region may encompass “SGGQQ,” “GAGQQ,” “GQGPY,” “AGQQ” or “SQ,” wherein some positions may be absent
  MISC_FEATURE (679)..(683): This region may encompass “SGGQQ,” “GAGQQ,” “GQGPY,” “AGQQ” or “SQ,” wherein some positions may be absent
  MISC_FEATURE (687)..(691): This region may encompass “SGGQQ,” “GAGQQ,” “GQGPY,” “AGQQ” or “SQ,” wherein some positions may be absent
  MISC_FEATURE (695)..(699): This region may encompass “SGGQQ,” “GAGQQ,” “GQGPY,” “AGQQ” or “SQ,” wherein some positions may be absent
  MISC_FEATURE (703)..(707): This region may encompass “SGGQQ,” “GAGQQ,” “GQGPY,” “AGQQ” or “SQ,” wherein some positions may be absent
  MISC_FEATURE (644)..(707): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (711)..(720): This region may encompass 6-10 residues
  MISC_FEATURE (727)..(731): This region may encompass “SGGQQ,” “GAGQQ,” “GQGPY,” “AGQQ” or “SQ,” wherein some positions may be absent
  MISC_FEATURE (735)..(739): This region may encompass “SGGQQ,” “GAGQQ,” “GQGPY,” “AGQQ” or “SQ,” wherein some positions may be absent
  MISC_FEATURE (743)..(747): This region may encompass “SGGQQ,” “GAGQQ,” “GQGPY,” “AGQQ” or “SQ,” wherein some positions may be absent
  MISC_FEATURE (751)..(755): This region may encompass “SGGQQ,” “GAGQQ,” “GQGPY,” “AGQQ” or “SQ,” wherein some positions may be absent
  MISC_FEATURE (759)..(763): This region may encompass “SGGQQ,” “GAGQQ,” “GQGPY,” “AGQQ” or “SQ,” wherein some positions may be absent
  MISC_FEATURE (767)..(771): This region may encompass “SGGQQ,” “GAGQQ,” “GQGPY,” “AGQQ” or “SQ,” wherein some positions may be absent
  MISC_FEATURE (775)..(779): This region may encompass “SGGQQ,” “GAGQQ,” “GQGPY,” “AGQQ” or “SQ,” wherein some positions may be absent
  MISC_FEATURE (783)..(787): This region may encompass “SGGQQ,” “GAGQQ,” “GQGPY,” “AGQQ” or “SQ,” wherein some positions may be absent
  MISC_FEATURE (724)..(787): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (791)..(800): This region may encompass 6-10 residues
  MISC_FEATURE (807)..(811): This region may encompass “SGGQQ,” “GAGQQ,” “GQGPY,” “AGQQ” or “SQ,” wherein some positions may be absent
  MISC_FEATURE (815)..(819): This region may encompass “SGGQQ,” “GAGQQ,” “GQGPY,” “AGQQ” or “SQ,” wherein some positions may be absent
  MISC_FEATURE (823)..(827): This region may encompass “SGGQQ,” “GAGQQ,” “GQGPY,” “AGQQ” or “SQ,” wherein some positions may be absent
  MISC_FEATURE (831)..(835): This region may encompass “SGGQQ,” “GAGQQ,” “GQGPY,” “AGQQ” or “SQ,” wherein some positions may be absent
  MISC_FEATURE (839)..(843): This region may encompass “SGGQQ,” “GAGQQ,” “GQGPY,” “AGQQ” or “SQ,” wherein some positions may be absent
  MISC_FEATURE (847)..(851): This region may encompass “SGGQQ,” “GAGQQ,” “GQGPY,” “AGQQ” or “SQ,” wherein some positions may be absent
  MISC_FEATURE (855)..(859): This region may encompass “SGGQQ,” “GAGQQ,” “GQGPY,” “AGQQ” or “SQ,” wherein some positions may be absent
  MISC_FEATURE (863)..(867): This region may encompass “SGGQQ,” “GAGQQ,” “GQGPY,” “AGQQ” or “SQ,” wherein some positions may be absent
  MISC_FEATURE (804)..(867): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (871)..(880): This region may encompass 6-10 residues
  MISC_FEATURE (887)..(891): This region may encompass “SGGQQ,” “GAGQQ,” “GQGPY,” “AGQQ” or “SQ,” wherein some positions may be absent
  MISC_FEATURE (895)..(899): This region may encompass “SGGQQ,” “GAGQQ,” “GQGPY,” “AGQQ” or “SQ,” wherein some positions may be absent
  MISC_FEATURE (903)..(907): This region may encompass “SGGQQ,” “GAGQQ,” “GQGPY,” “AGQQ” or “SQ,” wherein some positions may be absent
  MISC_FEATURE (911)..(915): This region may encompass “SGGQQ,” “GAGQQ,” “GQGPY,” “AGQQ” or “SQ,” wherein some positions may be absent
  MISC_FEATURE (919)..(923): This region may encompass “SGGQQ,” “GAGQQ,” “GQGPY,” “AGQQ” or “SQ,” wherein some positions may be absent
  MISC_FEATURE (927)..(931): This region may encompass “SGGQQ,” “GAGQQ,” “GQGPY,” “AGQQ” or “SQ,” wherein some positions may be absent
  MISC_FEATURE (935)..(939): This region may encompass “SGGQQ,” “GAGQQ,” “GQGPY,” “AGQQ” or “SQ,” wherein some positions may be absent
  MISC_FEATURE (943)..(947): This region may encompass “SGGQQ,” “GAGQQ,” “GQGPY,” “AGQQ” or “SQ,” wherein some positions may be absent
  MISC_FEATURE (884)..(947): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (951)..(960): This region may encompass 6-10 residues
  MISC_FEATURE (967)..(971): This region may encompass “SGGQQ,” “GAGQQ,” “GQGPY,” “AGQQ” or “SQ,” wherein some positions may be absent
  MISC_FEATURE (975)..(979): This region may encompass “SGGQQ,” “GAGQQ,” “GQGPY,” “AGQQ” or “SQ,” wherein some positions may be absent
  MISC_FEATURE (983)..(987): This region may encompass “SGGQQ,” “GAGQQ,” “GQGPY,” “AGQQ” or “SQ,” wherein some positions may be absent
  MISC_FEATURE (991)..(995): This region may encompass “SGGQQ,” “GAGQQ,” “GQGPY,” “AGQQ” or “SQ,” wherein some positions may be absent
  MISC_FEATURE (999)..(1003): This region may encompass “SGGQQ,” “GAGQQ,” “GQGPY,” “AGQQ” or “SQ,” wherein some positions may be absent
  MISC_FEATURE (1007)..(1011): This region may encompass “SGGQQ,” “GAGQQ,” “GQGPY,” “AGQQ” or “SQ,” wherein some positions may be absent
  MISC_FEATURE (1015)..(1019): This region may encompass “SGGQQ,” “GAGQQ,” “GQGPY,” “AGQQ” or “SQ,” wherein some positions may be absent
  MISC_FEATURE (1023)..(1027): This region may encompass “SGGQQ,” “GAGQQ,” “GQGPY,” “AGQQ” or “SQ,” wherein some positions may be absent
  MISC_FEATURE (964)..(1027): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1031)..(1040): This region may encompass 6-10 residues
  MISC_FEATURE (1047)..(1051): This region may encompass “SGGQQ,” “GAGQQ,” “GQGPY,” “AGQQ” or “SQ,” wherein some positions may be absent
  MISC_FEATURE (1055)..(1059): This region may encompass “SGGQQ,” “GAGQQ,” “GQGPY,” “AGQQ” or “SQ,” wherein some positions may be absent
  MISC_FEATURE (1063)..(1067): This region may encompass “SGGQQ,” “GAGQQ,” “GQGPY,” “AGQQ” or “SQ,” wherein some positions may be absent
  MISC_FEATURE (1071)..(1075): This region may encompass “SGGQQ,” “GAGQQ,” “GQGPY,” “AGQQ” or “SQ,” wherein some positions may be absent
  MISC_FEATURE (1079)..(1083): This region may encompass “SGGQQ,” “GAGQQ,” “GQGPY,” “AGQQ” or “SQ,” wherein some positions may be absent
  MISC_FEATURE (1087)..(1091): This region may encompass “SGGQQ,” “GAGQQ,” “GQGPY,” “AGQQ” or “SQ,” wherein some positions may be absent
  MISC_FEATURE (1095)..(1099): This region may encompass “SGGQQ,” “GAGQQ,” “GQGPY,” “AGQQ” or “SQ,” wherein some positions may be absent
  MISC_FEATURE (1103)..(1107): This region may encompass “SGGQQ,” “GAGQQ,” “GQGPY,” “AGQQ” or “SQ,” wherein some positions may be absent
  MISC_FEATURE (1044)..(1107): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1111)..(1120): This region may encompass 6-10 residues
  MISC_FEATURE (1127)..(1131): This region may encompass “SGGQQ,” “GAGQQ,” “GQGPY,” “AGQQ” or “SQ,” wherein some positions may be absent
  MISC_FEATURE (1135)..(1139): This region may encompass “SGGQQ,” “GAGQQ,” “GQGPY,” “AGQQ” or “SQ,” wherein some positions may be absent
  MISC_FEATURE (1143)..(1147): This region may encompass “SGGQQ,” “GAGQQ,” “GQGPY,” “AGQQ” or “SQ,” wherein some positions may be absent
  MISC_FEATURE (1151)..(1155): This region may encompass “SGGQQ,” “GAGQQ,” “GQGPY,” “AGQQ” or “SQ,” wherein some positions may be absent
  MISC_FEATURE (1159)..(1163): This region may encompass “SGGQQ,” “GAGQQ,” “GQGPY,” “AGQQ” or “SQ,” wherein some positions may be absent
  MISC_FEATURE (1167)..(1171): This region may encompass “SGGQQ,” “GAGQQ,” “GQGPY,” “AGQQ” or “SQ,” wherein some positions may be absent
  MISC_FEATURE (1175)..(1179): This region may encompass “SGGQQ,” “GAGQQ,” “GQGPY,” “AGQQ” or “SQ,” wherein some positions may be absent
  MISC_FEATURE (1183)..(1187): This region may encompass “SGGQQ,” “GAGQQ,” “GQGPY,” “AGQQ” or “SQ,” wherein some positions may be absent
  MISC_FEATURE (1124)..(1187): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1191)..(1200): This region may encompass 6-10 residues
  MISC_FEATURE (1207)..(1211): This region may encompass “SGGQQ,” “GAGQQ,” “GQGPY,” “AGQQ” or “SQ,” wherein some positions may be absent
  MISC_FEATURE (1215)..(1219): This region may encompass “SGGQQ,” “GAGQQ,” “GQGPY,” “AGQQ” or “SQ,” wherein some positions may be absent
  MISC_FEATURE (1223)..(1227): This region may encompass “SGGQQ,” “GAGQQ,” “GQGPY,” “AGQQ” or “SQ,” wherein some positions may be absent
  MISC_FEATURE (1231)..(1235): This region may encompass “SGGQQ,” “GAGQQ,” “GQGPY,” “AGQQ” or “SQ,” wherein some positions may be absent
  MISC_FEATURE (1239)..(1243): This region may encompass “SGGQQ,” “GAGQQ,” “GQGPY,” “AGQQ” or “SQ,” wherein some positions may be absent
  MISC_FEATURE (1247)..(1251): This region may encompass “SGGQQ,” “GAGQQ,” “GQGPY,” “AGQQ” or “SQ,” wherein some positions may be absent
  MISC_FEATURE (1255)..(1259): This region may encompass “SGGQQ,” “GAGQQ,” “GQGPY,” “AGQQ” or “SQ,” wherein some positions may be absent
  MISC_FEATURE (1263)..(1267): This region may encompass “SGGQQ,” “GAGQQ,” “GQGPY,” “AGQQ” or “SQ,” wherein some positions may be absent
  MISC_FEATURE (1204)..(1267): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1271)..(1280): This region may encompass 6-10 residues
  MISC_FEATURE (1287)..(1291): This region may encompass “SGGQQ,” “GAGQQ,” “GQGPY,” “AGQQ” or “SQ,” wherein some positions may be absent
  MISC_FEATURE (1295)..(1299): This region may encompass “SGGQQ,” “GAGQQ,” “GQGPY,” “AGQQ” or “SQ,” wherein some positions may be absent
  MISC_FEATURE (1303)..(1307): This region may encompass “SGGQQ,” “GAGQQ,” “GQGPY,” “AGQQ” or “SQ,” wherein some positions may be absent
  MISC_FEATURE (1311)..(1315): This region may encompass “SGGQQ,” “GAGQQ,” “GQGPY,” “AGQQ” or “SQ,” wherein some positions may be absent
  MISC_FEATURE (1319)..(1323): This region may encompass “SGGQQ,” “GAGQQ,” “GQGPY,” “AGQQ” or “SQ,” wherein some positions may be absent
  MISC_FEATURE (1327)..(1331): This region may encompass “SGGQQ,” “GAGQQ,” “GQGPY,” “AGQQ” or “SQ,” wherein some positions may be absent
  MISC_FEATURE (1335)..(1339): This region may encompass “SGGQQ,” “GAGQQ,” “GQGPY,” “AGQQ” or “SQ,” wherein some positions may be absent
  MISC_FEATURE (1343)..(1347): This region may encompass “SGGQQ,” “GAGQQ,” “GQGPY,” “AGQQ” or “SQ,” wherein some positions may be absent
  MISC_FEATURE (1284)..(1347): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1351)..(1360): This region may encompass 6-10 residues
  MISC_FEATURE (1367)..(1371): This region may encompass “SGGQQ,” “GAGQQ,” “GQGPY,” “AGQQ” or “SQ,” wherein some positions may be absent
  MISC_FEATURE (1375)..(1379): This region may encompass “SGGQQ,” “GAGQQ,” “GQGPY,” “AGQQ” or “SQ,” wherein some positions may be absent
  MISC_FEATURE (1383)..(1387): This region may encompass “SGGQQ,” “GAGQQ,” “GQGPY,” “AGQQ” or “SQ,” wherein some positions may be absent
  MISC_FEATURE (1391)..(1395): This region may encompass “SGGQQ,” “GAGQQ,” “GQGPY,” “AGQQ” or “SQ,” wherein some positions may be absent
  MISC_FEATURE (1399)..(1403): This region may encompass “SGGQQ,” “GAGQQ,” “GQGPY,” “AGQQ” or “SQ,” wherein some positions may be absent
  MISC_FEATURE (1407)..(1411): This region may encompass “SGGQQ,” “GAGQQ,” “GQGPY,” “AGQQ” or “SQ,” wherein some positions may be absent
  MISC_FEATURE (1415)..(1419): This region may encompass “SGGQQ,” “GAGQQ,” “GQGPY,” “AGQQ” or “SQ,” wherein some positions may be absent
  MISC_FEATURE (1423)..(1427): This region may encompass “SGGQQ,” “GAGQQ,” “GQGPY,” “AGQQ” or “SQ,” wherein some positions may be absent
  MISC_FEATURE (1364)..(1427): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1431)..(1440): This region may encompass 6-10 residues
  MISC_FEATURE (1447)..(1451): This region may encompass “SGGQQ,” “GAGQQ,” “GQGPY,” “AGQQ” or “SQ,” wherein some positions may be absent
  MISC_FEATURE (1455)..(1459): This region may encompass “SGGQQ,” “GAGQQ,” “GQGPY,” “AGQQ” or “SQ,” wherein some positions may be absent
  MISC_FEATURE (1463)..(1467): This region may encompass “SGGQQ,” “GAGQQ,” “GQGPY,” “AGQQ” or “SQ,” wherein some positions may be absent
  MISC_FEATURE (1471)..(1475): This region may encompass “SGGQQ,” “GAGQQ,” “GQGPY,” “AGQQ” or “SQ,” wherein some positions may be absent
  MISC_FEATURE (1479)..(1483): This region may encompass “SGGQQ,” “GAGQQ,” “GQGPY,” “AGQQ” or “SQ,” wherein some positions may be absent
  MISC_FEATURE (1487)..(1491): This region may encompass “SGGQQ,” “GAGQQ,” “GQGPY,” “AGQQ” or “SQ,” wherein some positions may be absent
  MISC_FEATURE (1495)..(1499): This region may encompass “SGGQQ,” “GAGQQ,” “GQGPY,” “AGQQ” or “SQ,” wherein some positions may be absent
  MISC_FEATURE (1503)..(1507): This region may encompass “SGGQQ,” “GAGQQ,” “GQGPY,” “AGQQ” or “SQ,” wherein some positions may be absent
  MISC_FEATURE (1444)..(1507): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1511)..(1520): This region may encompass 6-10 residues
  MISC_FEATURE (1527)..(1531): This region may encompass “SGGQQ,” “GAGQQ,” “GQGPY,” “AGQQ” or “SQ,” wherein some positions may be absent
  MISC_FEATURE (1535)..(1539): This region may encompass “SGGQQ,” “GAGQQ,” “GQGPY,” “AGQQ” or “SQ,” wherein some positions may be absent
  MISC_FEATURE (1543)..(1547): This region may encompass “SGGQQ,” “GAGQQ,” “GQGPY,” “AGQQ” or “SQ,” wherein some positions may be absent
  MISC_FEATURE (1551)..(1555): This region may encompass “SGGQQ,” “GAGQQ,” “GQGPY,” “AGQQ” or “SQ,” wherein some positions may be absent
  MISC_FEATURE (1559)..(1563): This region may encompass “SGGQQ,” “GAGQQ,” “GQGPY,” “AGQQ” or “SQ,” wherein some positions may be absent
  MISC_FEATURE (1567)..(1571): This region may encompass “SGGQQ,” “GAGQQ,” “GQGPY,” “AGQQ” or “SQ,” wherein some positions may be absent
  MISC_FEATURE (1575)..(1579): This region may encompass “SGGQQ,” “GAGQQ,” “GQGPY,” “AGQQ” or “SQ,” wherein some positions may be absent
  MISC_FEATURE (1583)..(1587): This region may encompass “SGGQQ,” “GAGQQ,” “GQGPY,” “AGQQ” or “SQ,” wherein some positions may be absent
  MISC_FEATURE (1524)..(1587): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1591)..(1600): This region may encompass 6-10 residues
  MISC_FEATURE (1)..(1600): This sequence may encompass 2-20 “GGY- [GPG-X1]n1-GPS-(A)n2” repeating units, wherein X1 is “SGGQQ,” “GAGQQ,” “GQGPY,” “AGQQ” or “SQ,” n1 is 4-8 and n2 is 6-10 and some positions may be absent
GGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAA

SEQ ID NO: 35 - protein, 5 aa, Artificial Sequence
  Description of Artificial Sequence Synthetic peptide
SGGQQ

SEQ ID NO: 36 - protein, 5 aa, Artificial Sequence
  Description of Artificial Sequence Synthetic peptide
GAGQQ

SEQ ID NO: 37 - protein, 5 aa, Artificial Sequence
  Description of Artificial Sequence Synthetic peptide
GQGPY

SEQ ID NO: 38 - protein, 4 aa, Artificial Sequence
  Description of Artificial Sequence Synthetic peptide
AGQQ

(19) United States
(12) Patent Application Publication
(10) Pub. No.: US 2019/0169242 A1
(43) Pub. Date: Jun. 6, 2019

(54) Methods of Generating Recombinant Spider Silk Protein Fibers

(71) Applicant: Bolt Threads, Inc., Emeryville, CA (US)

(72) Inventors: Maxime Boulet-Audet, El Cerrito, CA (US); Gregory Wilson Rice, South San Francisco, CA (US); Krystle Marie Moody, Sausalito, CA (US); Jamie McAusland Bainbridge, Berkeley, CA (US); Steven Tom, Berkeley, CA (US); Amir Ahmad Bakhtiary Davijani, Oakland, CA (US); Lindsay Wray, Benicia, CA (US)

(21) Appl. No.: 16/176,939

(22) Filed: Oct. 31, 2018

Related U.S. Application Data
(60) Provisional application No. 62/579,789, filed on Oct. 31, 2017.

(51) Int. Cl. C07K 14/435; D01D 5/06; D01F 6/68; D02G 3/02; D01D 5/24; A61K 9/70

(52) U.S. Cl. CPC C07K 14/43586; D01D 5/06; C07K 14/43518; A61K 9/70; D02G 3/02; D01D 5/24; D01F 6/68

(57) ABSTRACT

Provided herein are scalable methods of generating a recombinant silk polypeptide with a coefficient of friction sufficient to form mechanical interactions with other fibers.

### CROSS-REFERENCE TO RELATED APPLICATIONS

This application claims the benefit of US Provisional Application No. 62/579,789, filed Oct. 31, 2017, the contents of which are incorporated by reference in its entirety.

### FIELD OF THE INVENTION

The present invention relates to scalable methods for producing fiber comprising recombinant spider silk polypeptides with a coefficient of friction sufficient to form a web of fibers, either by itself or in a blend with other fibers.

### BACKGROUND OF THE INVENTION

Protein-based materials are of increasing interest as an alternative to petroleum-based products. To this end, considerable effort has been made to develop methods of making materials and fibers from regenerated protein sources derived from plants (e.g., zein, soy, wheat gluten) and animals (e.g., casein, keratin and collagen). Fiber made from regenerated protein dates back to the 1890s and has been made using various traditional wet-spinning techniques.

Silk proteins such as silk fibroin and spidroins have a complex tertiary structure which make them an ideal candidate for the creation of protein-based materials. Specifically, silk proteins form complex beta sheet structures that are extremely stable and only denature at very high temperatures, far above the melting temperature of the protein.

For many textile applications, it is desirable to have fibers that are capable of imparting a mechanical or frictional force on other fibers to form a web of fibers (hereinafter “fiberweb”). While there has been significant work performed in generating fibers and materials from recombinant spider silk polypeptides using traditional spinning and molding processes (see U.S. Pat. No. 7,057,023), much of this work has been proof-of-principle work that is not reproducible or scalable for mass commercialization. Further, none of this work has looked at scalable mechanisms for generating silk that has frictional or mechanical properties which would cause it to form a fiberweb. Accordingly, there is a need for scalable processes of manufacturing fiber from recombinant spider silk polypeptides which can form fiberwebs.

### SUMMARY OF THE INVENTION

Provided herein, according to some embodiments of the invention, is a drawn fiber comprising recombinant spider silk polypeptide, wherein the fiber is generated by: dissolving a powder comprising the recombinant spider silk polypeptide into a solvent to generate a spin dope; extruding the spin dope into a coagulation bath to form a precursor fiber; and subjecting the precursor fiber to a turbulent air source, wherein the fiber has a mean coefficient of friction greater than 0.60.

Also provided herein, according to some embodiments, is a comprising recombinant spider silk polypeptide, wherein the plurality of fibers are generated by: dissolving a powder comprising a recombinant spider silk polypeptide into a solvent to generate a spin dope; extruding the spin dope into a coagulation bath to form a plurality of precursor fibers; and subjecting the plurality of precursor fibers to a turbulent air source, thereby generating the plurality of fibers, wherein one or more of the plurality of fibers has a mean coefficient of friction that is greater than 0.60.

In some embodiments, the plurality of fibers is an unfused plurality of fibers. In some embodiments, the plurality of fibers forms a sufficient mechanical interaction with wool to form a web of fibers.

In some embodiments, one or more of the plurality of fibers has a mean coefficient of friction that is greater than 0.70. In some embodiments, one or more of the plurality of fibers has a mean coefficient of friction that is greater than 0.80. In some embodiments, one or more of the plurality of fibers has a mean coefficient of friction that is greater than 0.85.

In some embodiments, the powder comprising the recombinant spider silk polypeptide is comprised of at least 55% recombinant spider silk polypeptide by weight.

In some embodiments, the mean coefficient of friction is measured using the ASTM 3808 standard. In some embodiments, the mean coefficient of friction is measured at intervals of 1cm along a length of at least 6 meters.

In some embodiments, the fiber is produced by a continuous wet-spinning process.

In some embodiments, the plurality of fibers comprises more than 30 fibers. In some embodiments, the plurality of fibers comprises more than 50 fibers.

In some embodiments, the plurality of fibers produces a loss in weight of less than 4.68% when carded with a blend of 60% wool by weight. In some embodiments, the plurality of fibers produces an overall loss in weight of less than 13% when processed into yarn with a blend of 60% wool by weight.

Also provided herein, according to some embodiments, is a staple yarn comprising wool and a plurality of fibers of divided into discrete lengths, wherein the plurality of fibers are generated by: dissolving a powder comprising a recombinant spider silk polypeptide into a solvent to generate a spin dope; extruding the spin dope into a coagulation bath to form a plurality of precursor fibers; and subjecting the plurality of precursor fibers to a turbulent air source, thereby generating the plurality of fibers, wherein one or more of the plurality of fibers has a mean coefficient of friction that is greater than 0.60.

Also provided herein, according to some embodiments, is a knitted garment comprising a staple yarn comprising wool and a plurality of fibers of divided into discrete lengths, wherein the plurality of fibers are generated by: dissolving a powder comprising a recombinant spider silk polypeptide into a solvent to generate a spin dope; extruding the spin dope into a coagulation bath to form a plurality of precursor fibers; and subjecting the plurality of precursor fibers to a turbulent air source, thereby generating the plurality of fibers, wherein one or more of the plurality of fibers has a mean coefficient of friction that is greater than 0.60.

Also provided herein, according to some embodiments, is a recombinant fiber comprising a recombinant polypeptide, wherein said fiber comprises a mean coefficient of friction of greater than 0.60.

In some embodiments, the fiber comprises a mean coefficient of friction of greater than 0.70, greater than 0.80, or greater than 0.85. In some embodiments, the mean coefficient of friction is measured using the ASTM 3808 standard. In some embodiments, the mean coefficient of friction is measured at intervals of 1cm along a length of at least 6 meters. In some embodiments, the recombinant polypeptide is a recombinant spider silk polypeptide.

In some embodiments, the recombinant polypeptide is a silk-like polypeptide.

In some embodiments, the recombinant polypeptide comprises repeat units, wherein each repeat unit has at least 95% sequence identity to a sequence that comprises from 2 to 20 quasi-repeat units, each quasi-repeat unit having a composition comprising {GGY-[GPG-X1]n1-GPS-(A)n2} (SEQ ID NO: 34), wherein for each quasi-repeat unit: X1 is independently selected from the group consisting of SGGQQ (SEQ ID NO: 35), GAGQQ (SEQ ID NO: 36), GQGPY (SEQ ID NO: 37), AGQQ (SEQ ID NO: 38), and SQ; and n1 is from 4 to 8, and n2 is from 6 to 10.

In some embodiments, n1 is from 4 to 5 for at least half of the quasi-repeat units. In some embodiments, n2 is from 5 to 8 for at least half of the quasi-repeat units. In some embodiments, each quasi-repeat unit has at least 95% sequence identity to a MaSp2 dragline silk protein subsequence. In some embodiments, the repeat unit comprises SEQ ID NO: 2. In some embodiments, the repeat comprises a block sequence selected from Table 1. In some embodiments, the recombinant polypeptide comprises SEQ ID NO: 1

Also provided herein, according to some embodiments, is a plurality of recombinant fibers comprising a recombinant polypeptide, wherein said fibers comprise a mean coefficient of friction of greater than 0.60.

In some embodiments, the plurality of fibers is an unfused plurality of fibers. In some embodiments, the plurality of fibers forms a sufficient mechanical interaction with wool to form a web of fibers. In some embodiments, the plurality of fibers comprises at least 30 fibers or at least 50 fibers.

Also provided herein, according to some embodiments, is a staple yarn comprising wool and a plurality of recombinant fibers divided into discrete lengths, wherein the plurality of recombinant fibers comprising a recombinant polypeptide, wherein said fibers comprise a mean coefficient of friction of greater than 0.60.

Also provided herein, according to some embodiments, is a knitted garment comprising a staple yarn comprising wool and a plurality of recombinant fibers divided into discrete lengths, wherein the plurality of recombinant fibers comprising a recombinant polypeptide, wherein said fibers comprise a mean coefficient of friction of greater than 0.60.

Also provided herein, according to some embodiments, is a knitted garment comprising a staple yarn comprising wool and a plurality of recombinant fibers divided into discrete lengths, wherein the plurality of recombinant fibers comprising a recombinant polypeptide, wherein said fibers comprise a mean coefficient of friction of greater than 0.60. Also provided herein, according to some embodiments, is a knitted garment comprising a staple yarn, wherein said staple yarn comprises wool and a plurality of recombinant fibers divided into discrete lengths, wherein said plurality of recombinant fibers comprise silk-like polypeptides, and wherein said plurality of recombinant fibers comprises a mean coefficient of friction of greater than 0.60. In some embodiments, the plurality of recombinant fibers are generated by: dissolving a powder comprising a recombinant spider silk polypeptide into a solvent to generate a spin dope; extruding the spin dope into a coagulation bath to form a plurality of precursor fibers; and subjecting the plurality of precursor fibers to a turbulent air source, thereby generating the plurality of recombinant fibers comprising the recombinant spider silk polypeptide.

Also provided herein, according to some embodiments, is a method of generating a fiber comprising recombinant spider silk polypeptide, comprising: dissolving a powder comprising a recombinant spider silk polypeptide into a solvent to generate a spin dope; extruding the spin dope into a coagulation bath to form a precursor fiber; and subjecting the precursor fiber to a turbulent air source, thereby generating the fiber comprising recombinant spider silk polypeptide, wherein the fiber comprising recombinant spider silk polypeptide has a mean coefficient of friction that is greater than 0.60.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 is a diagram of components and processes used to generate high-friction fibers, according to an embodiment of the invention.

FIG. 2 is a 200× scanning electron microscopy image of a low-friction fiber.

FIG. 3 is a 200× scanning electron microscopy image of a high-friction fiber generated using turbulent air flow according to an embodiment of the invention.

FIG. 4 is a high magnification (600×) scanning electron microscopy image of a high-friction fiber generated using turbulent air flow, according to an embodiment of the invention.

### DETAILED DESCRIPTION OF THE INVENTION

The details of various embodiments of the invention are set forth in the description below. Other features, objects, and advantages of the invention will be apparent from the description. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology-A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and claims, the word “comprise” or variations such as “comprises” or “comprising,” will be understood to imply the inclusion of a stated integer or group of integers but not the exclusion of any other integer or group of integers.

The term “wet spinning” as used herein refers to a method of forming fibers from a polymer wherein the polymer is dissolved in solution and extruded into a substance that makes the dissolved polymer coagulate.

The term “coagulation bath” as used herein refers to a liquid bath comprising a substance that makes fibers coagulate.

The term “drawing” as used herein with reference to a fiber refers to the application of force to stretch a wet-spun fiber along its longitudinal axis after extrusion of the fiber into a coagulation bath. The term “undrawn fibers” refers to fibers that have been extruded into a coagulation bath but have not been subject to any drawing force. The term “draw ratio” is a term of art commonly defined as the ratio between the collection rate and the feeding rate. At constant volume, it can be determine from a ratio of the initial diameter (Dᵢ) and final diameter (D_{f}) of the fiber (i.e., Dᵢ/D_{f}).

The term “glass transition temperature” as used herein refers to the temperature at which a substance transitions from a hard, rigid or “glassy” state into a more pliable, “rubbery” state.

The term “melting temperature” as used herein refers to the temperature at which a substance transitions from a rubbery state to a less-ordered liquid phase. As used herein, the term melting temperature does not refer to the temperature at which recombinant proteins containing beta sheets are denatured.

The term “plasticizer” as used herein refers to any molecule that interacts with a polypeptide sequence to prevent the polypeptide sequence from forming tertiary structures and bonds and/or to increase the mobility of the polypeptide sequence.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Overview

Provided herein are scalable methods of post-processing wet-spun fiber comprising recombinant silk proteins. These techniques discussed herein employ the use of a turbulent air source to create a fiber that has a sufficient coefficient of friction to form mechanical interactions with other similar fibers or different types of fibers (e.g. wool). These techniques are designed to be scalable and therefore optimal for the large-scale production of recombinant silk fibers.

### Recombinant Silk Proteins

The present disclosure describes embodiments of the invention including fibers synthesized from synthetic proteinaceous copolymers (i.e., recombinant polypeptides). Suitable proteinaceous co-polymers are discussed in U.S. Patent Publication No. 2016/0222174, published Aug. 45, 2016, U.S. Patent Publication No. 2018/0111970, published Apr. 26, 2018, and U.S. Patent Publication No. 2018/0057548, published Mar. 1, 2018, each of which are incorporated by reference herein in its entirety.

In some embodiments, the synthetic proteinaceous copolymers are made from silk-like polypeptide sequences. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful molded body compositions by secretion from an industrially scalable microorganism. Large (approximately 40 kDa to approximately 100 kDa) block copolymer polypeptides engineered from silk repeat domain fragments, including sequences from almost all published amino acid sequences of spider silk polypeptides, can be expressed in the modified microorganisms described herein. In some embodiments, silk polypeptide sequences are matched and designed to produce highly expressed and secreted polypeptides.

In some embodiments, block copolymers are engineered from a combinatorial mix of silk polypeptide domains across the silk polypeptide sequence space. In some embodiments, the block copolymers are made by expressing and secreting in scalable organisms (e.g., yeast, fungi, and gram positive bacteria). In some embodiments, the block copolymer polypeptide comprises 0 or more N-terminal domains (NTD), 1 or more repeat domains (REP), and 0 or more C-terminal domains (CTD). In some aspects of the embodiment, the block copolymer polypeptide is >100 amino acids of a single polypeptide chain. In some embodiments, the block copolymer polypeptide comprises a domain that is at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of a block copolymer polypeptide as disclosed in International Publication No. WO/2015/042164, “Methods and Compositions for Synthesizing Improved Silk Fibers,” incorporated by reference in its entirety.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example: Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block co-polymer polypeptides having sequence similarity and/or amino acid composition similarity to the repeat domains of native silk proteins can be used to manufacture consistent fibers, textiles and other articles of manufacture as described herein.

In some embodiments, a list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of generating fibers, textiles and other articles of manufacture as described herein.

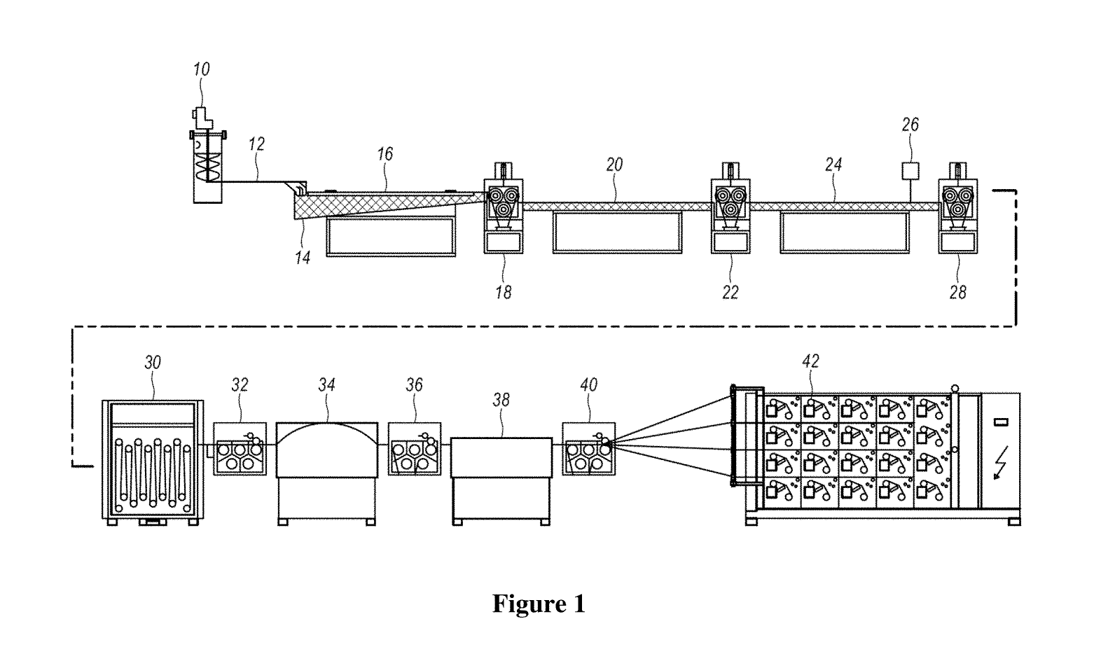

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture, as depicted in FIG. 1. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. In some embodiments, block sequences comprise a glycine rich region followed by a polyA region. In some embodiments, short (˜1-10) amino acid motifs appear multiple times inside of blocks. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG (SEQ ID NO: 31) is, for the purposes of the present invention, the same as GSGAG (SEQ ID NO: 32) and the same as GGSGA (SEQ ID NO: 33); they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1
Samples of Block Sequences

| | Silk | Representative Block | SEQ ID |
|---|---|---|---|
| Species | Type | Amino Acid Sequence | NO: |
| Aliatypus | Fibroin 1 | GAASSSSTIITTKSASASAAADASAAATASAASRSSANA | 4 |
| gulosus | | AASAFAQSFSSILLESGYFCSIFGSSISSSYAAAIASAA | |
| | | SRAAAESNGYTTHAYACAKAVASAVERVTSGADAYAYAQ | |
| | | AISDALSHALLYTGRLNTANANSLASAFAYAFANAAAQA | |
| | | SASSASAGAASASGAASASGAGSAS | |
| Plectreurys | Fibroin 1 | GAGAGAGAGAGAGAGAGSGASTSVSTSSSSGSGAGAGAG | 5 |
| tristis | | SGAGSGAGAGSGAGAGAGAGGAGAGFGSGLGLGYGVGLS | |
| | | SAQAQAQAQAAAQAQAQAQAQAYAAAQAQAQAQAQAQAA | |
| | | AAAAAAAAA | |
| Plectreurys | Fibroin 4 | GAAQKQPSGESSVATASAAATSVTSGGAPVGKPGVPAPI | 6 |
| tristis | | FYPQGPLQQGPAPGPSNVQPGTSQQGPIGGVGGSNAFSS | |
| | | SFASALSLNRGFTEVISSASATAVASAFQKGLAPYGTAF | |
| | | ALSAASAAADAYNSIGSGANAFAYAQAFARVLYPLVQQY | |
| | | GLSSSAKASAFASAIASSFSSGTSGQGPSIGQQQPPVTI | |
| | | SAASASAGASAAAVGGGQVGQGPYGGQQQSTAASASAAA | |
| | | ATATS | |
| Araneus | TuSp | GNVGYQLGLKVANSLGLGNAQALASSLSQAVSAVGVGAS | 7 |
| gemmoides | | SNAYANAVSNAVGQVLAGQGILNAANAGSLASSFASALS | |
| | | SSAASVASQSASQSQAASQSQAAASAFRQAASQSASQSD | |
| | | SRAGSQSSTKTTSTSTSGSQADSRSASSSASQASASAFA | |
| | | QQSSASLSSSSSFSSAFSSATSISAV | |
| Argiope | TuSp | GSLASSFASALSASAASVASSAAAQAASQSQAAASAFSR | 8 |
| aurantia | | AASQSASQSAARSGAQSISTTTTTSTAGSQAASQSASSA | |
| | | ASQASASSFARASSASLAASSSFSSAFSSANSLSALGNV | |
| | | GYQLGFNVANNLGIGNAAGLGNALSQAVSSVGVGASSST | |
| | | YANAVSNAVGQFLAGQGILNAANA | |
| Deinopis | TuSp | GASASAYASAISNAVGPYLYGLGLFNQANAASFASSFAS | 9 |
| spinosa | | AVSSAVASASASAASSAYAQSAAAQAQAASSAFSQAAAQ | |
| | | SAAAASAGASAGAGASAGAGAVAGAGAVAGAGAVAGASA | |
| | | AAASQAAASSSASAVASAFAQSASYALASSSAFANAFAS | |
| | | ATSAGYLGSLAYQLGLTTAYNLGLSNAQAFASTLSQAVT | |
| | | GVGL | |
| Nephila | TuSp | GATAASYGNALSTAAAQFFATAGLLNAGNASALASSFAR | 10 |
| clavipes | | AFSASAESQSFAQSQAFQQASAFQQAASRSASQSAAEAG | |
| | | STSSSTTTTTSAARSQAASQSASSSYSSAFAQAASSSLA | |
| | | TSSALSRAFSSVSSASAASSLAYSIGLSAARSLGIADAA | |
| | | GLAGVLARAAGALGQ | |
| Argiope | Flag | GGAPGGGPGGAGPGGAGFGPGGGAGFGPGGGAGFGPGGA | 11 |
| trifasciata | | AGGPGGPGGPGGPGGAGGYGPGGAGGYGPGGVGPGGAGG | |
| | | YGPGGAGGYGPGGSGPGGAGPGGAGGEGPVTVDVDVTVG | |
| | | PEGVGGGPGGAGPGGAGFGPGGGAGFGPGGAPGAPGGPG | |
| | | GPGGPGGPGGPGGVGPGGAGGYGPGGAGGVGPAGTGGFG | |
| | | PGGAGGFGPGGAGGFGPGGAGGFGPAGAGGYGPGGVGPG | |
| | | GAGGFGPGGVGPGGSGPGGAGGEGPVTVDVDVSV | |
| Nephila | Flag | GVSYGPGGAGGPYGPGGPYGPGGEGPGGAGGPYGPGGVG | 12 |
| clavipes | | PGGSGPGGYGPGGAGPGGYGPGGSGPGGYGPGGSGPGGY | |
| | | GPGGSGPGGYGPGGSGPGGYGPGGYGPGGSGPGGSGPGG | |
| | | SGPGGYGPGGTGPGGSGPGGYGPGGSGPGGSGPGGYGPG | |
| | | GSGPGGFGPGGSGPGGYGPGGSGPGGAGPGGVGPGGFGP | |
| | | GGAGPGGAAPGGAGPGGAGPGGAGPGGAGPGGAGPGGAG | |
| | | PGGAGGAGGAGGSGGAGGSGGTTIIEDLDITIDGADGPI | |
| | | TISEELPISGAGGSGPGGAGPGGVGPGGSGPGGVGPGGS | |
| | | GPGGVGPGGSGPGGVGPGGAGGPYGPGGSGPGGAGGAGG | |
| | | PGGAYGPGGSYGPGGSGGPGGAGGPYGPGGEGPGGAGGP | |
| | | YGPGGAGGPYGPGGAGGPYGPGGEGGPYGP | |
| Latrodectus | AcSp | GINVDSDIGSVTSLILSGSTLQMTIPAGGDDLSGGYPGG | 13 |
| hesperus | | FPAGAQPSGGAPVDFGGPSAGGDVAAKLARSLASTLASS | |
| | | GVFRAAFNSRVSTPVAVQLTDALVQKIASNLGLDYATAS | |
| | | KLRKASQAVSKVRMGSDTNAYALAISSALAEVLSSSGKV | |
| | | ADANINQIAPQLASGIVLGVSTTAPQFGVDLSSINVNLD | |
| | | ISNVARNMQASIQGGPAPITAEGPDFGAGYPGGAPTDLS | |
| | | GLDMGAPSDGSRGGDATAKLLQALVPALLKSDVFRAIYK | |
| | | RGTRKQVVQYVTNSALQQAASSLGLDASTISQLQTKATQ | |
| | | ALSSVSADSDSTAYAKAFGLAIAQVLGTSGQVNDANVNQ | |
| | | IGAKLATGILRGSSAVAPRLGIDLS | |
| Argiope | AcSp | GAGYTGPSGPSTGPSGYPGPLGGGAPFGQSGFGGSAGPQ | 14 |
| trifasciata | | GGFGATGGASAGLISRVANALANTSTLRTVLRTGVSQQI | |
| | | ASSVVQRAAQSLASTLGVDGNNLARFAVQAVSRLPAGSD | |
| | | TSAYAQAFSSALFNAGVLNASNIDTLGSRVLSALLNGVS | |
| | | SAAQGLGINVDSGSVQSDISSSSSFLSTSSSSASYSQAS | |
| | | ASSTS | |
| Uloborus | AcSp | GASAADIATAIAASVATSLQSNGVLTASNVSQLSNQLAS | 15 |
| diversus | | YVSSGLSSTASSLGIQLGASLGAGFGASAGLSASTDISS | |
| | | SVEATSASTLSSSASSTSVVSSINAQLVPALAQTAVLNA | |
| | | AFSNINTQNAIRIAELLTQQVGRQYGLSGSDVATASSQI | |
| | | RSALYSVQQGSASSAYVSAIVGPLITALSSRGVVNASNS | |
| | | SQIASSLATAILQFTANVAPQFGISIPTSAVQSDLSTIS | |
| | | QSLTAISSQTSSSVDSSTSAFGGISGPSGPSPYGPQPSG | |
| | | PTFGPGPSLSGLTGFTATFASSFKSTLASSTQFQLIAQS | |
| | | NLDVQTRSSLISKVLINALSSLGISASVASSIAASSSQS | |
| | | LLSVSA | |
| Euprosthenops | MaSp1 | GGQGGQGQGRYGQGAGSSAAAAAAAAAAAAAA | 16 |
| australis | | | |
| Tetragnatha | MaSp1 | GGLGGGQGAGQGGQQGAGQGGYGSGLGGAGQGASAAAAA | 17 |
| kauaiensis | | AAA | |
| Argiope | MaSp2 | GGYGPGAGQQGPGSQGPGSGGQQGPGGLGPYGPSAAAAA | 18 |
| aurantia | | AAA | |
| Deinopis | MaSp2 | GPGGYGGPGQQGPGQGQYGPGTGQQGQGPSGQQGPAGAA | 19 |
| spinosa | | AAAAAAA | |
| Nephila | MaSp2 | GPGGYGLGQQGPGQQGPGQQGPAGYGPSGLSGPGGAAAA | 20 |
| clavata | | AAA | |
| Deinopis | MiSp | GAGYGAGAGAGGGAGAGTGYGGGAGYGTGSGAGYGAGVGY | 21 |
| Spinosa | | GAGAGAGGGAGAGAGGGTGAGAGGGAGAGYGAGTGYGAGA | |
| | | GAGGGAGAGAGAGAGAGAGAGSGAGAGYGAGAGYGAGAGA | |
| | | GGVAGAGAAGGAGAAGGAGAAGGAGAAGGAGAGAGAGSGA | |
| | | GAGAGGGARAGAGG | |
| Latrodectus | MiSp | GGGYGRGQGAGAGVGAGAGAAAGAAAIARAGGYGQGAGGY | 22 |
| hesperus | | GQGQGAGAAAGAAAGAGAGGYGQGAGGYGRGQGAGAGAGA | |
| | | GAGARGYGQGAGAGAAAGAAASAGAGGYGQGAGGYGQGQG | |
| | | AGAAAGAAASAGAGGYGQGAGGYGQGQGA | |
| Nephila | MiSp | GAGAGGAGYGRGAGAGAGAAAGAGAGAAAGAGAGAGGYGG | 23 |
| clavipes | | QGGYGAGAGAGAAAAAGAGAGGAAGYSRGGRAGAAGAGAG | |
| | | AAAGAGAGAGGYGGQGGYGAGAGAGAAAAAGAGSGGAGGY | |
| | | GRGAGAGAAAGAGAAAGAGAGAGGYGGQGGYGAGAGAAAA | |
| | | A | |
| Nephilengys | MiSp | GAGAGVGGAGGYGSGAGAGAGAGAGAASGAAAGAAAGAGA | 24 |
| cruentata | | GGAGGYGTGQGYGAGAGAGAGAGAGGAGGYGRGAGAGAGA | |
| | | GAGGAGGYGAGQGYGAGAGAGAAAAAGDGAGAGGAGGYGR | |
| | | GAGAGAGAGAAAGAGAGGAGGYGAGQGYGAGAGAGAAAGA | |
| | | GAGGAGGYGAGQGYGAGAGAGAAAAA | |
| Uloborus | MiSp | GSGAGAGSGYGAGAGAGAGSGYGAGSSASAGSAINTQTVT | 25 |
| diversus | | SSTTTSSQSSAAATGAGYGTGAGTGASAGAAASGAGAGYG | |
| | | GQAGYGQGAGASARAAGSGYGAGAGAAAAAGSGYGAGAGA | |
| | | GAGSGYGAGAAA | |
| Uloborus | MiSp | GAGAGYRGQAGYIQGAGASAGAAAAGAGVGYGGQAGYGQG | 26 |
| diversus | | AGASAGAAAAAGAGAGRQAGYGQGAGASAGAAAAGAGAGR | |
| | | QAGYGQGAGASAGAAAAGADAGYGGQAGYGQGAGASAGAA | |
| | | ASGAGAGYGGQAGYGQGAGASAGAAAAGAGAGYLGQAGYG | |
| | | QGAGASAGAAAGAGAGYGGQAGYGQGTGAAASAAASSA | |
| Araneus | MaSp1 | GGQGGQGGYGGLGSQGAGQGGYGAGQGAAAAAAAAGGAGG | 27 |
| ventricosus | | AGRGGLGAGGAGQGYGAGLGGQGGAGQAAAAAAAGGAGGA | |
| | | RQGGLGAGGAGQGYGAGLGGQGGAGQGGAAAAAAAAGGQG | |
| | | GQGGYGGLGSQGAGQGGYGAGQGGAAAAAAAAGGQGGQGG | |
| | | YGGLGSQGAGQGGYGGRQGGAGAAAAAAAA | |
| Dolomedes | MaSp1 | GGAGAGQGSYGGQGGYGQGGAGAATATAAAAGGAGSGQGG | 28 |
| tenebrosus | | YGGQGGLGGYGQGAGAGAAAAAAAAAGGAGAGQGGYGGQG | |
| | | GQGGYGQGAGAGAAAAAAGGAGAGQGGYGGQGGYGQGGGA | |
| | | GAAAAAAAASGGSGSGQGGYGGQGGLGGYGQGAGAGAGAA | |
| | | ASAAAA | |
| Nephilengys | MaSp | GGAGQGGYGGLGGQGAGAAAAAAGGAGQGGYGGQGAGQGA | 29 |
| cruentata | | AAAAASGAGQGGYEGPGAGQGAGAAAAAAGGAGQGGYGGL | |
| | | GGQGAGQGAGAAAAAAGGAGQGGYGGLGGQGAGQGAGAAA | |
| | | AAAGGAGQGGYGGQGAGQGAAAAAAGGAGQGGYGGLGSGQ | |
| | | GGYGRQGAGAAAAAAAA | |
| Nephilengys | MaSp | GGAGQGGYGGLGGQGAGAAAAAAGGAGQGGYGGQGAGQGA | 30 |
| cruentata | | AAAAASGAGQGGYGGPGAGQGAGAAAAAAGGAGQGGYGGL | |
| | | GGQGAGQGAGAAAAAAGGAGQGGYGGQGAGQGAAAAAAGG | |
| | | AGQGGYGGLGSGQGGYGGQGAGAAAAAGGAGQGGYGGLGG | |
| | | QGAGQGAGAAAAAA | |

Fiber-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers or molded bodies include natural amino acid sequence information and other modifications described herein. The repeat domain is decomposed into repeat sequences containing representative blocks (usually 1-8 depending upon the type of silk) that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3× FLAG nucleotide coding sequence expressing the 3× FLAG polypeptide sequence: DYKDDDDKDYKDDDDKDYKDDDDK (SEQ ID NO: 3). In some embodiments, the silk polypeptide nucleotide coding sequence is operatively linked to other affinity tags such as 6-8 His residues.

In some embodiments, the recombinant spider silk polypeptides are based on recombinant spider silk protein fragment sequences derived from MaSp2, such as from the species Argiope bruennichi. In some embodiments, the synthesized fiber contains protein molecules that include two to twenty repeat units, in which a molecular weight of each repeat unit is greater than about 20 kDa. Within each repeat unit of the copolymer are more than about 60 amino acid residues, often in the range 60 to 100 amino acids that are organized into a number of “quasi-repeat units.” In some embodiments, the repeat unit of a polypeptide described in this disclosure has at least 95% sequence identity to a MaSp2 dragline silk protein sequence.

The repeat unit of the proteinaceous block copolymer that forms fibers with good mechanical properties can be synthesized using a portion of a silk polypeptide. These polypeptide repeat units contain alanine-rich regions and glycine-rich regions, and are 150 amino acids in length or longer. Some exemplary sequences that can be used as repeats in the proteinaceous block copolymers of this disclosure are provided in in co-owned PCT Publication WO 2015/042164, incorporated by reference in its entirety, and were demonstrated to express using a Pichia expression system.

In some embodiments, the spider silk protein comprises: at least two occurrences of a repeat unit, the repeat unit comprising: more than 150 amino acid residues and having a molecular weight of at least 10 kDa; an alanine-rich region with 6 or more consecutive amino acids, comprising an alanine content of at least 80%; a glycine-rich region with 12 or more consecutive amino acids, comprising a glycine content of at least 40% and an alanine content of less than 30%; and wherein the fiber comprises at least one property selected from the group consisting of a modulus of elasticity greater than 550 cN/tex, an extensibility of at least 10% and an ultimate tensile strength of at least 15 cN/tex.

In some embodiments, wherein the recombinant spider silk protein comprises repeat units wherein each repeat unit has at least 95% sequence identity to a sequence that comprises from 2 to 20 quasi-repeat units; each quasi-repeat unit comprises {GGY-[GPG-X1]n1-GPS-(A)n2} (SEQ ID NO: 34), wherein for each quasi-repeat unit: X1 is independently selected from the group consisting of SGGQQ (SEQ ID NO: 35), GAGQQ (SEQ ID NO: 36), GQGPY (SEQ ID NO: 37), AGQQ (SEQ ID NO: 38), and SQ; and n1 is from 4 to 8, and n2 is from 6-10. The repeat unit is composed of multiple quasi-repeat units.

In some embodiments, 3 “long” quasi repeats are followed by 3 “short” quasi-repeat units. As mentioned above, short quasi-repeat units are those in which n1=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

In some embodiments, the recombinant spider silk polypeptide comprises the polypeptide sequence of SEQ ID NO: 1 (i.e., 18B). In some embodiments, the repeat unit is a polypeptide comprising SEQ ID NO: 2. These sequences are provided in Table 2:

TABLE 2
Exemplary polypeptides sequences
of recombinant protein and repeat unit

| SEQ ID | Polypeptide Sequence |
|---|---|
| SEQ ID | GGYGPGAGQQGPGSGGQQGPGGQGPYGSGQQGPGGAGQQG |
| NO: 1 | PGGQGPYGPGAAAAAAAAAGGYGPGAGQQGPGGAGQQGPG |
| | SQGPGGQGPYGPGAGQQGPGSQGPGSGGQQGPGGQGPYGP |
| | SAAAAAAAAAGGYGPGAGQRSQGPGGQGPYGPGAGQQGPG |
| | SQGPGSGGQQGPGGQGPYGPSAAAAAAAAGGYGPGAGQQG |
| | PGSQGPGSGGQQGPGGQGPYGPGAAAAAAAVGGYGPGAGQ |
| | QGPGSQGPGSGGQQGPGGQGPYGPSAAAAAAAAGGYGPGA |
| | GQQGPGSQGPGSGGQQGPGGQGPYGPSAAAAAAAAGGYGP |
| | GAGQQGPGSGGQQGPGGQGPYGSGQQGPGGAGQQGPGGQG |
| | PYGPGAAAAAAAAAGGYGPGAGQQGPGGAGQQGPGSQGPG |
| | GQGPYGPGAGQQGPGSQGPGSGGQQGPGGQGPYGPSAAAA |
| | AAAAAGGYGPGAGQRSQGPGGQGPYGPGAGQQGPGSQGPG |
| | SGGQQGPGGQGPYGPSAAAAAAAAGGYGPGAGQQGPGSQG |
| | PGSGGQQGPGGQGPYGPGAAAAAAAVGGYGPGAGQQGPGS |
| | QGPGSGGQQGPGGQGPYGPSAAAAAAAAGGYGPGAGQQGP |
| | GSQGPGSGGQQGPGGQGPYGPSAAAAAAAAGGYGPGAGQQ |
| | GPGSGGQQGPGGQGPYGSGQQGPGGAGQQGPGGQGPYGPG |
| | AAAAAAAAAGGYGPGAGQQGPGGAGQQGPGSQGPGGQGPY |
| | GPGAGQQGPGSQGPGSGGQQGPGGQGPYGPSAAAAAAAAA |
| | GGYGPGAGQRSQGPGGQGPYGPGAGQQGPGSQGPGSGGQQ |
| | GPGGQGPYGPSAAAAAAAAGGYGPGAGQQGPGSQGPGSGG |
| | QQGPGGQGPYGPGAAAAAAAVGGYGPGAGQQGPGSQGPGS |
| | GGQQGPGGQGPYGPSAAAAAAAAGGYGPGAGQQGPGSQGP |
| | GSGGQQGPGGQGPYGPSAAAAAAAA |
| SEQ ID | GGYGPGAGQQGPGSGGQQGPGGQGPYGSGQQGPGGAGQQG |
| NO: 2 | PGGQGPYGPGAAAAAAAAAGGYGPGAGQQGPGGAGQQGPG |
| | SQGPGGQGPYGPGAGQQGPGSQGPGSGGQQGPGGQGPYGP |
| | SAAAAAAAAAGGYGPGAGQRSQGPGGQGPYGPGAGQQGPG |
| | SQGPGSGGQQGPGGQGPYGPSAAAAAAAAGGYGPGAGQQG |
| | PGSQGPGSGGQQGPGGQGPYGPGAAAAAAAVGGYGPGAGQ |
| | QGPGSQGPGSGGQQGPGGQGPYGPSAAAAAAAAGGYGPGA |
| | GQQGPGSQGPGSGGQQGPGGQGPYGPSAAAAAAAA |

In some embodiments, the structure of fibers formed from the described recombinant spider silk polypeptides form beta-sheet structures, beta-turn structures, or alpha-helix structures. In some embodiments, the secondary, tertiary and quaternary protein structures of the formed fibers are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. While not wishing to be bound by theory, the structural properties of the proteins within the spider silk are theorized to be related to fiber mechanical properties. Crystalline regions in a fiber have been linked with the tensile strength of a fiber, while the amorphous regions have been linked to the extensibility of a fiber. The major ampullate (MA) silks tend to have higher strengths and less extensibility than the flagelliform silks, and likewise the MA silks have higher volume fraction of crystalline regions compared with flagelliform silks. Furthermore, theoretical models based on the molecular dynamics of crystalline and amorphous regions of spider silk proteins, support the assertion that the crystalline regions have been linked with the tensile strength of a fiber, while the amorphous regions have been linked to the extensibility of a fiber. Additionally, the theoretical modeling supports the importance of the secondary, tertiary and quaternary structure on the mechanical properties of RPFs. For instance, both the assembly of nano-crystal domains in a random, parallel and serial spatial distributions, and the strength of the interaction forces between entangled chains within the amorphous regions, and between the amorphous regions and the nano-crystalline regions, influenced the theoretical mechanical properties of the resulting fibers.

In some embodiments, the molecular weight of the silk protein may range from 20 kDa to 2000 kDa, or greater than 20 kDa, or greater than 10 kDa, or greater than 5 kDa, or from 5 to 400 kDa, or from 5 to 300 kDa, or from 5 to 200 kDa, or from 5 to 100 kDa, or from 5 to 50 kDa, or from 5 to 500 kDa, or from 5 to 1000 kDa, or from 5 to 2000 kDa, or from 10 to 400 kDa, or from 10 to 300 kDa, or from 10 to 200 kDa, or from 10 to 100 kDa, or from 10 to 50 kDa, or from 10 to 500 kDa, or from 10 to 1000 kDa, or from 10 to 2000 kDa, or from 20 to 400 kDa, or from 20 to 300 kDa, or from 20 to 200 kDa, or from 40 to 300 kDa, or from 40 to 500 kDa, or from 20 to 100 kDa, or from 20 to 50 kDa, or from 20 to 500 kDa, or from 20 to 1000 kDa, or from 20 to 2000 kDa.

### Characterization of Recombinant Spider Silk Polypeptide Powder Impurities

Different recombinant spider silk polypeptides have different physiochemical properties such as melting temperature and glass transition temperature based on the strength and stability of the secondary and tertiary structures formed by the proteins. Silk polypeptides form beta sheet structures in a monomeric form. In the presence of other monomers, the silk polypeptides form a three-dimensional crystalline lattice of beta sheet structures. The beta sheet structures are separated from, and interspersed with, amorphous regions of polypeptide sequences.

Beta sheet structures are extremely stable at high temperatures—the melting temperature of beta-sheets is approximately 257° C. as measured by fast scanning calorimetry. See Cebe et al., Beating the Heat—Fast Scanning Melts Silk Beta Sheet Crystals, Nature Scientific Reports 3:1130 (2013). As beta sheet structures are thought to stay intact above the glass transition temperature of silk polypeptides, it has been postulated that the structural transitions seen at the glass transition temperature of silk polypeptides are due to increased mobility of the amorphous regions between the beta sheets.

Plasticizers lower the glass transition temperature and the melting temperature of silk proteins by increasing the mobility of the amorphous regions and potentially disrupting beta sheet formation. Suitable plasticizers used for this purpose include water, polyalcohols (polyols) and urea. As hydrophilic portions of silk polypeptides can bind ambient water present in the air as humidity, bound ambient water may plasticize silk polypeptides.

In addition, in instances where recombinant spider silk polypeptides are produced by fermentation and recovered as recombinant spider silk polypeptide powder from the same, there may be impurities present in the recombinant spider silk polypeptide powder that act as plasticizers or otherwise inhibit the formation of tertiary structures. For example, residual lipids and sugars may act as plasticizers and thus influence the glass transition temperature of the protein by interfering with the formation of tertiary structures.

Various well-established methods may be used to assess the purity and relative composition of recombinant spider silk polypeptide powder. Size Exclusion Chromatography separates molecules based on their relative size and can be used to analyze the relative amounts of recombinant spider silk polypeptide in its aggregate and monomeric forms as well as the amount of high, low and intermediate molecular weight impurities in the recombinant spider silk polypeptide powder. Similarly, Rapid High Performance Liquid Chromatography may be used to measure various compounds present in a solution such as monomeric forms of the recombinant spider silk polypeptide. Ion Exchange Liquid Chromatography may be used to assess the concentrations of various trace molecules in solution, including impurities such as lipids and sugars. Other methods of chromatography and quantification of various molecules such as mass spectrometry are well established in the art.

Depending on the embodiment, the recombinant spider silk polypeptide powder may have a purity calculated based on the amount of the recombinant spider silk polypeptide in is monomeric and aggregate forms by weight relative to the other components of the powder. In various instances, the purity can range from 50% by weight to 90% by weight, depending on the type of recombinant spider silk polypeptide and the techniques used to recover, separate and post-process the recombinant spider silk polypeptide powder.

### Dope Rheology and Secondary Structures

Rheology is commonly used in fiber spinning to analyze the physio-chemical characteristics of material that is spun into fiber such as polymers. Different rheological characteristics may impact the ability to spin material into fiber and the mechanical characteristics of the spun fiber. Rheology can be also used to indirectly study the secondary and tertiary structures formed by recombinant spider silk polypeptides under different temperatures and conditions. Depending on the embodiment, shear rheometers and/or extensional rheometers may be used to analyze different rheological properties by oscillatory and extensional rheology.

In some embodiments, small amplitude oscillatory shear (SAOS) rheology is used to measure various rheological properties including but not limited to the loss tangent (G″/G′), complex viscosity (η*) and phase angle (δ). In these embodiments, a SAOS rheometer outputs a stress response as a function of oscillation frequency, ω, which can be broken down into elastic and viscous contributions. The elastic component, the solid-like behavior, is measured by the storage modulus (or elastic modulus), G′(ω), while the viscous component, the fluid-like behavior, is measured by loss modulus (or viscous modulus), G″(ω). The rheometer also measures the ratio of G″/G′, called the loss tangent, or tan (δ), which describes the extent to which the complex fluid is liquid-like (tan (δ)>>1) or solid-like (tan (δ)<<1). The rheometer outputs the values of the phase angle, δ, which spans from 90° (ideal liquid) to 0° (ideal solid). At G′ and G″ crossover, δ is 45°, and the material is transitioning from being more liquid-like to more solid-like. In addition, the complex viscosity η*, defined by η*=G*(ω)/ω, is also measured by the rheometer. In embodiments where the silk polypeptide is a recombinant silk protein that is wet spun into fiber, different rheological characteristics such as complex viscosity, loss tangent, and phase angle may be assessed based on a spin dope comprising the recombinant spider silk polypeptide dissolved into an appropriate solvent. Similarly, in embodiments where the silk polypeptide is melt spun into fiber, different rheological characteristics may be assessed based on a spin dope comprising the silk polypeptide and a plasticizer.

Depending on the embodiment, various rheology metrics may be used to determine whether a spin dope comprising recombinant spider silk polypeptide is suitable for wet spinning. For example, in some embodiments, a complex viscosity as measured at 10 Hz of less than 30 Pa s, less than 25 Pa s, less than 20 Pa s, less than 15 Pa S can indicate that a spin dope comprising recombinant spider silk polypeptide is not suitable for wet spinning. Similarly, in some embodiments, a complex viscosity as measured at 10 Hz of higher than 70 Pa S, higher than 75 Pa S, higher than 80 Pa S, higher than 85 Pa S can indicate that a spin dope comprising recombinant spider silk polypeptide is not suitable for wet spinning. In some embodiments, the phase angle of the spin dope comprising recombinant spider silk polypeptide may between 50-90°, 55-85°, 65-85°, 65-80°, 70-80°, 70-85°, 65-70°, or 50-65°.

In some embodiments, Differential Scanning calorimetry is used to determine the glass transition temperature of the recombinant spider silk polypeptide and/or fiber containing the same. In a specific embodiment, Modulated Differential Scanning calorimetry is used to measure the glass transition temperature.

Depending on the embodiment and the type of recombinant spider silk polypeptide, the glass transition temperature may have range of values. However, a measured glass transition temperature that is much lower that is typically observed for a recombinant spider silk polypeptide in its solid form may indicate that impurities or the presence of other plasticizers.

In addition, Fourier Transform Infrared (FTIR) spectroscopy data may be combined with rheology data to provide both direct characterization of tertiary structures in the recombinant silk powder and/or spin dope containing the same. FTIR can be used to quantify secondary structures in silk polypeptides and/or dope comprising the silk polypeptides as discussed below in the section entitled “Fourier Transform Infrared (FTIR) Spectroscopy.”

Depending on the embodiment, FTIR may be used to quantify beta-sheet structures present in the recombinant spider silk polypeptide powder and/or spin dope containing the same. In addition, in some embodiments, FTIR may be used to quantify impurities such as sugars and lipids present in the recombinant spider silk polypeptide powder. However, various chaotropes and solubilizers used in different protein pre-processing methods may diminish the number of tertiary structures in recombinant spider silk polypeptide powder or spin dope containing the same. Accordingly, there may be no correspondence between the amount of beta sheet structures in recombinant spider silk polypeptide powder before and after is it spun into fiber. Similarly, there may be little to no correspondence between the glass transition temperature of a powder before and after it is spun into fiber.

In some embodiments, rheological data characterizing the recombinant spider silk polypeptides may be combined with FTIR to analyze secondary and tertiary structures formed in by the polypeptides. In a specific embodiment, rheological data may be captured in conjunction with FTIR spectra. For exemplary methods of combining rheology and FTIR, see Boulet-Audet et al., Silk protein aggregation kinetics revealed by Rheo-IR, Acta Biomaterialia 10:776-784(2014), the entirety of which is herein incorporated by reference.

Similarly, various methods of characterizing impurities in the recombinant silk powder may be combined with rheological and/or FTIR data to analyze the relationship between the presence of impurities and the formation of tertiary structures.

### Wet Spinning Recombinant Silk Proteins

Depending on the embodiment, the recombinant spider silk polypeptides may be wet-spun into fiber using various established methods. Exemplary methods of wet-spinning recombinant spider silk polypeptides are discussed in detail in U.S. Pat. No. 7,057,023, the entirety of which is herein incorporated by reference.

In most wet spinning embodiments, recombinant spider silk polypeptides are dissolved to form a spin dope. Suitable solvents for use in a spin dope include but are not limited to formic acid, aqueous solutions (e.g., eADF4), dimethyl sulfoxide (DMSO), N-methyl morpholine N-oxide (NMMO), N, N-dimethylformamide (DMF), hexafluoroisopropanol (HFIP), hexafluoroacetone hydrate, trifluoroacetic acid, water, phosphoric acid and any combination thereof. Other suitable solvents are listed in Koeppel and Holland, Progress and Trends in Artificial Silk Spinning: A Systematic Review, ACS Biomater. Sci. Eng. 3:226-237 (2017), the entirety of which is herein incorporated by reference. Depending on the solvent used, various salts may be added to the spin dope.

In various embodiments, the concentration of solvent and recombinant silk protein in the spin dope may be varied based on the properties of the silk polypeptide and the type of solvent used. Concentrations may be adjusted in part based on rheological data such as the complex viscosity or the phase angle. In specific embodiments where formic acid is used to dissolve the 18B protein (SEQ ID NO: 1), suitable concentrations of recombinant silk protein by weight in the spin dope range from: 20-60% by weight, 20-50% by weight, 20-40% by weight, 30-40% by weight, 30-60% by weight, or 30-50% by weight.

In some embodiments, a plasticizer will be added to the spin dope. Suitable plasticizers include water, polyols (e.g glycerol), lactic acid, methyl hydroperoxide, ascorbic acid, 1,4-dihydroxybenzene (1,4 Benzenediol) Benzene-1,4-diol, phosphoric acid, ethylene glycol, propylene glycol, triethanolamine, acid acetate, propane-1,3-diol or any combination thereof.

In some embodiments, various agents may be added to the spin dope to alter the rheological characteristics of the spin dope such as elongational viscosity, shear viscosity and linear viscoelasticity. Suitable agents used to alter the elongational viscosity include polyethylene glycol (PEG), Tween, Sodium dodecyl sulfate, polyethylene oxide, or any combination thereof. Other suitable agents are well known in the art.

In various embodiments, the spin dope may be subject to mixing or agitation to ensure a homogeneous spin dope. Suitable methods of mixing the spin dope include but are not limited to: centrifugal mixers, high-shear mixers, and twin screw mixing. Other mixing methods are well established in the art.

In some embodiments, a pigment or dye may be added to the spin dope to perform “solution dying” of the fiber. Solution dying is an optimal method of dying fiber as it eliminates large amounts of wastewater involved in dying finished fibers and textiles. Dyes may form covalent or cationic bonds with the recombinant silk protein to provide a colorfast fiber. Any type of dye and/or pigment may be used for solution dying including but not limited to cationic dyes, anionic dyes, zwitterionic dyes and dispersions of pigment.

Depending on the required initial denier of the extruded fiber, spin dope comprising the recombinant silk protein may be extruded through spinnerets with varying orifice sizes. In most embodiments, the orifice will range from 50-200 μm, 50-100 μm, 50-150 μm, 100-150 μm, 100-200 μm or 150-200 μm. In some embodiments, the ideal orifice size will be based on the final draw ratio of the fiber. For example, a higher initial denier of an extruded fiber may be subject to a higher draw ratio than a smaller initial denier extruded fiber

In various embodiments, different coagulation baths may be used, alone or sequentially. In some embodiments, alcohol such as ethanol or methanol will be used as a coagulation agent to precipitate or otherwise coagulated the extruded spin dope. Suitable alcohols for this purpose include ethanol, methanol or any combination thereof. For example, suitable coagulation bath could contain 80% ethanol and 20% methanol; 60% ethanol and 40% methanol; 40% ethanol and 60% methanol; or 20% ethanol and 80% methanol.

In some embodiments, the coagulation bath will combine a coagulation agent with the solvent used to generate the spin dope. For example, in some embodiments, the coagulation bath will comprise an alcohol and formic acid. In a specific embodiment, the coagulation bath will comprise 90% ethanol and 10% formic acid.

In some embodiments, the coagulation bath will combine a coagulation agent with a plasticizer such as water or any of the plasticizers listed above. In a specific embodiment, the coagulation bath will comprise 90% ethanol and 10% H₂0.

Depending on the embodiment, the fiber may be subject to any number of coagulation baths, in any order. In some embodiments, the fiber may be the following series of coagulation baths: a coagulation bath comprising the solvent used for dope spinning, a coagulation bath comprising only alcohol, and a coagulation bath comprising a plasticizer.

Depending on the embodiment, the total residence time in the one or more coagulation baths will range from 20-50 seconds, from 25-50 seconds, from 30-50 seconds, from 35-50 seconds, from 40-50 seconds, from 20-40 seconds, from 20-35 seconds, from 20-30 seconds, and/or from 30-40 seconds. In most embodiments, the residence time will be sufficient to eliminate most or all residual formic acid from the fiber.

In most embodiments of the present invention, the extruded fiber will not be subject to any drawing while it is transferred through the one or more coagulation baths. In other words, the extruded fiber will only be subject to the minimal amount of force necessary to move the fiber through the coagulation bath and collect fiber on the godets. Extruded fiber that is not subject to any drawing force is herein referred to as “precursor fiber.”

### Using Air Flow to Dry Fibers

In some embodiments, a multi-orifice spinneret may be used to concurrently wet spin a plurality of fibers (also referred to herein as a “tow of fibers”). Depending on the embodiment, the number of orifices in the spinneret and the corresponding number of fibers in the tow of fibers can range from 5-100, 20-100, 20-80, 30-80, 20-60.

In most embodiments of the present invention, the precursor fiber(s) will be subject to an air flow in order to dry the precursor fiber(s) as it exits the coagulation bath. Subjecting the fiber(s) to an airflow can cause plasticizers such as water and any alcohol present in the fiber(s) to evaporate from the fiber(s). In some embodiments, the air flow will be a turbulent air flow. In other embodiments, the air flow will be laminar or non-turbulent. Many different types or airflows may be combined in any order to dry the precursor fiber(s).

In some embodiments, an air flow may be used to add a degree of roughness to the fiber which, in turn, can enhance the coefficient of friction of the fiber(s). Fiber(s) with a sufficient coefficient of friction can form mechanical interactions with themselves or other types of fibers (e.g. wool) to form fiberwebs. In some embodiments, a turbulent air flow may be used to create a coefficient of friction that is variable over the length of the fiber(s).

In some embodiments, a turbulent or non-turbulent air flow may be employed to prevent a plurality of fibers from fusing to one another. A plurality of fibers that are from as separate fibers that are not fused to each other is herein referred to as an “unfused” plurality of fibers.

Depending on the embodiments, the coefficient of friction of the fiber can be measured in different ways. In a specific embodiment, the coefficient of friction will be measured according to the ASTM 3808 standard. The coefficient of friction and/or roughness of the fiber may also be visually assessed using microscopy.

As discussed above, the coefficient of friction of the fiber impacts the ability of the fiber to form mechanical interactions (i.e. entanglement) with other fibers to form a fiberweb. The ability of the fiber to interact with other fibers to form a fiberweb may be measured in several different ways. Carding is the mechanical process used to disentangle and intermix fiber into a continuous fiberweb. Fiber that does not have the ability to form a fiberweb through carding is extruded from the carding machine as waste product. Accordingly, one method of assessing the utility of a fiber for forming a fiberweb is to assess the amount of waste that is produced by the carding process.

In other embodiments, the utility of a fiber for forming a fiberweb may be measured by determining the thickness of the fiberweb extruded from the carding machine. In other embodiments, the fiberweb may be assessed using microscopy. Suitable methods of assessing the utility of a fiber in forming a fiberweb are discussed in detail in Doguc et al., Influence of Fiber Type on Fiberweb Properties in High-Speed Carding, International Nonwovens Journal, 13(2):48-53 (2004) the entirety of which is herein incorporated by reference.

Fiber that is capable of carding and forming a fiberweb may be used to create staple or spun yarns. A staple yarn is a yarn that is comprised a number of fibers that have a limited fixed length. Staple yarn is created by taking the fiberweb that is output from the carding process (referred to as “sliver”) and then twisting the fiber into yarn. Depending on the embodiment, there may be a number of post-processing steps use to process the sliver such as pin drafting or combing the sliver. The staple yarn can then be used to form different garments or in other knitted objects (e.g. upholstery).

### Drawing Undrawn Fiber Over a Hot Surface

Precursor fiber may be also drawn in order to increase the orientation of the fiber and promote three-dimensional crystalline structure. The application of force in drawing promotes molecules to align on the axis of the fiber. Polymeric molecules such as polypeptides are partially aligned when forced to flow through the spinneret orifice.

In the present invention, the alignment is optimized by passing the precursor fiber over a uniform hot surface while the fiber is drawn. The term “hot surface” as used herein refers to a surface that has provides both a substantially uniform heat and a substantially uniform surface. Using a hot surface as a heat source eliminates variability seen using ambient heat sources, resulting in greater uniformity in results and consequent scalability of the process for commercial mass production of the fiber. In some embodiments, the hot surface will be a metal bar or surface. In other embodiments, the hot surface may be made of ceramic or other materials. Depending on the embodiment, the hot surface can be curved or otherwise configured to facilitate the fiber moving over the hot surface.

In embodiments of the present invention, the undrawn extruded fiber is simultaneously moved over the hot surface in contact with the hot surface as it is drawn. Depending on the embodiment, the temperature of the hot surface can range from 160-210° C., 180-210° C., 190-210° C., 195-210° C., 195-205° C., or 200-205° C.

Depending on the embodiment, the undrawn precursor fiber can be subject to different draw ratios while it is drawn over the hot surface. Depending on the embodiment, the draw ratio may range from 2 to 7. In some embodiments, the maximum stable draw ratio may depend on the temperature of the hot surface.

In some embodiments, the temperature of the hot surface is calculated as a function of the glass transition temperature of the undrawn precursor fiber. For example, the temperature of the hot surface can be calculated to be greater than 5° C., 10° C., 15° C., 20° C., or 25° C. greater than the glass transition temperature of the recombinant silk protein powder and/or the undrawn extruded fiber.

Depending on the embodiment and the rate at which fiber is passed over the uniform hot surface (referred to herein as the “reel rate”), the hot surface can vary in length (i.e. the size in cm of the hot surface that the fiber is drawn over), thus changing the duration of time that the undrawn precusor fiber is subject to heat and deformation. In most embodiments, the width of the hot bar will be no less than 1 cm. However, in various embodiments the width of the hot surface can range from 1-50 cm, 1-2 cm, 1-3 cm, 1-5 cm, 5-38cm, 38-50cm. Depending on the embodiment, the reel rate can range from 1 to 60 meters a minute.

Depending on the reel rate and the length of the hot surface, the total residence time over the hot surface may vary. In most embodiments the total residence time can range from 0.2 seconds to 3 seconds.

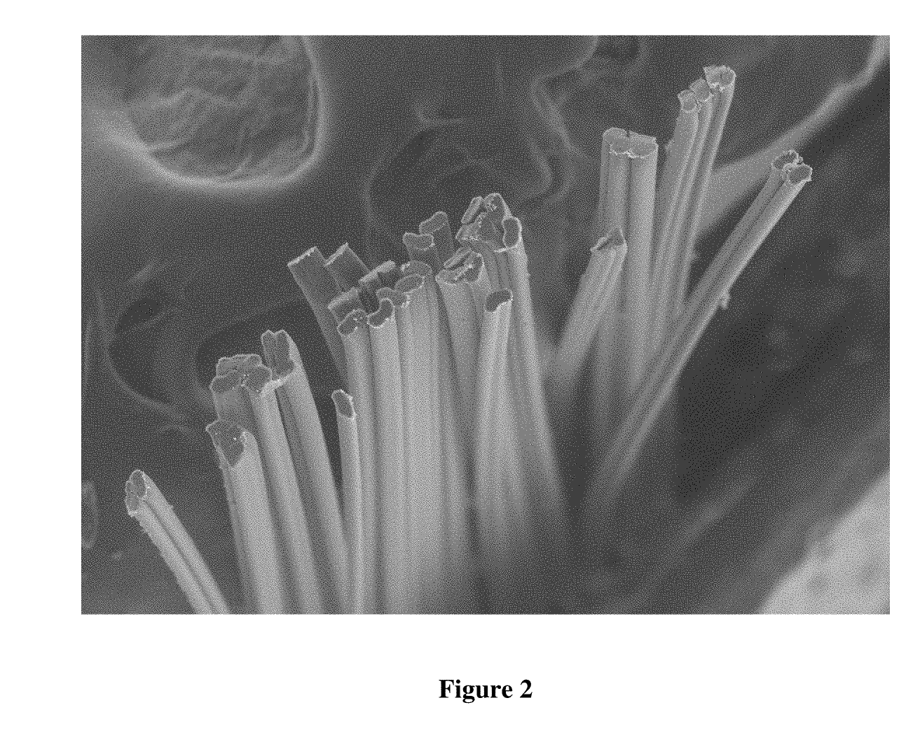

In addition, the undrawn fiber may be subject to varying force which provides different draw ratios. In most embodiments, the tensile force will be provided by godets as illustrated in FIGS. 2A and 2B. Depending on the embodiment, the godets will be positioned 40 cm to 300 cm (3 m) apart. In some embodiments, the godets will be placed such that the fiber that is passed over the hot surface is at an angle relative to the hot surface. For example, in instances where the hot surface is curved, the godets may be placed such that the fiber that is passed over the hot surface contact the hot surface and pivots over the hot surface at an angle of 20 to 60 degrees relative to the hot surface (see FIG. 2B).

In various embodiments, the deformation rate (i.e., the amount of deformation that the fiber is subject to with heat and drawing) of the undrawn fiber can vary based on the above factors. Deformation rate may be calculated based on the rate that the undrawn fiber is fed to the hot surface and the rate that the fiber is collected from the hot surface. For example, the fiber may be fed to the hot surface at a rate of 1 meters/minute and collected from the hot surface at a rate of 5 meters/minute. In a specific embodiment, the deformation rate E(t) is calculated using the following equation, where the rate that the fiber is fed to the hot surface is represented v₁, the rate that the fiber is collected from the hot surface is v2 and the length the deformation takes place over is L₀:

$$\dot{ϵ} ( t ) = \frac{{v}_{2} - {v}_{1}}{{L}_{0}} \qquad Equation 1$$

Depending on the embodiment, drawing over a hot surface may be performed in one step or multiple (i.e. two, three, or four) steps. Parameters such as the strain rate, the deformation rate, the reel rate, the temperature of the hot surface and the length of the hot surface may be varied or otherwise differ at each step. Performing drawing over multiple steps may affect the overall strain rate of the fiber, which may enhance formation of crystalline beta-sheet structures.

In some embodiments, drawing over a hot surface may be performed in conjunction with the use of an air flow to create fibers that are rough or have a desirable coefficient of friction.

### EXAMPLE 1

### Purity of Recombinant 18B Polypeptide Powder

18B polypeptide sequences (SEQID NO: 1) bound to a C-terminal 3× FLAG tag (SEQ ID NO: 3) (i.e., “18B-FLAG”) were produced through various lots of large-scale fermentation, recovered and dried in powders (“18B powder”). Exemplary lots of 18B powder used to create the fibers discussed in this section are indicated in the table below in the column entitled “Source Ref.”

Reverse Phase High Performance Liquid Chromatography (“RP-HPLC”) was used to measure the amount by weight of 18B polypeptide monomer in the powder. The various lots of powder were dissolved using a 5M Guanidine Thiocyanate (GdSCN) reagent and injected onto an Agilent Poroshell 300SB C3 2.1×75mm 5 μm column to separate constituents on the basis of hydrophobicity. The detection modality was UV absorbance of peptide bond at 215 nm (360 nm reference). The sample concentration of 18B-FLAG monomer was determined by using a lot of 18B-FLAG powder standard, for which the 18B-FLAG monomer concentration had been previously determined using Size Exclusion Chromatography (SEC-HPLC).

Table 3 (below) lists the purity of the exemplary lots of powder used. As shown below, the purity as expressed in % weight

TABLE 3
Exemplary and Average 18B monomer
by weight as determined by RP-HPLC

| Lot | 18B Monomer (Mass %) |
|---|---|
| 142 | 57.93 |
| 143 | 62.66 |
| 144 | 60.14 |
| 145 | 52.10 |
| 146 | 60.84 |
| 147 | 57.86 |
| 148 | 58.95 |
| 149 | 65.16 |
| 150 | 62.83 |
| Average | 58.83 |

### EXAMPLE 2

### Spin Dope Preparation and Rheology of the Spin Dopes

The above-discussed samples of 18B powder were dissolved in formic acid and mixed using a Thinky Planetary Centrifugal Mixer 400ARE-TWIN at 1600 RPM to generate spin dopes. Prior to dissolution, the 18B powder was baked to reduce the moisture content of the powder down to less than 4%.

A Malvern Kinexus Lab+Rotational Rheometer was used to measure the complex viscosity and the phase angle of the spin dopes. Parameters were set to a temperature of 22° C., a frequency of 100-0.1 Hz, and a strain of 1%. An interval of 3 points/decade was used to determine an average value for a given frequency.

Table 4 below includes the concentration by weight of the 18B powder in the spin dope, the complex viscosity and the phase angle as measured at 10 Hz. Data was not collected for 125-FACU.

TABLE 4
18B Powder Concentration in Spin Dope,
Complex Viscosity and Phase Angle

| | Dope | Complex | Phase Angle |
|---|---|---|---|
| | Concentration | Viscosity at | at 10 Hz |
| Lot | (wt %) | 10 Hz (Pas) | (°) |
| 142 | 36 | 48.71 | 69.12 |
| 143 | 36 | 43.99 | 68.16 |
| 144 | 36 | 58.98 | 65.41 |
| 145 | 36 | 54.65 | 68.33 |
| 146 | 36 | 62.76 | 65.37 |
| 147 | 36 | 67.24 | 65.38 |
| 148 | 36 | 42.67 | 65.26 |
| 149 | 36 | 65.17 | 66.33 |
| 150 | 36 | 62.45 | 69.94 |
| Average | 36 | 56.29 | 67.03 |

### EXAMPLE 3

### Spinning Conditions for Low-Friction Fibers

18 was wet-spun into fiber using traditional wet-spinning techniques. A spin dope was prepared using 67% formic acid (by weight) and 33% 18B powder (by weight). The spin dope was mixed using a FlackTek SpeedMixer DAC 600.2 VAC-LR vacuum mixer.

The spin dope was extruded directly into a coagulation bath comprised of 100% ethanol at room temperature through a spinneret orifice that is 50 μm in diameter at a rate of 1.25 ml/minute to form a precursor fiber. Both the spinneret and the coagulation bath were maintained at room temperature. Precursor fiber is then collected on a set of uptake godets at a reel rate of 3.2 meters/minute. The precursor fiber was then drawn between the uptake godets and a heated godet spaced 81 inches apart. The reel rate of the heated godet was 19.8 meters/minute, providing a draw ratio of 6.19×. The drawn fiber was then drawn between the heated godet and a final godet that were spaced 139 inches apart. The uptake rate of the final godet was 22 meters/minute providing a draw ration of 1.12×.

Between the heated godet and the final godet, the drawn fiber was passed through a 40-inch tube furnace that was heated to 200° C. A lubricant comprising 1% Setol® by weight in ethanol was applied to the drawn, heat-treated fiber at a rate of 1.1 mL/minute. The low-friction fiber was then wound on a spool for analysis.

### EXAMPLE 4

### Spinning Conditions for High-Friction Fibers

To produce high-friction fibers, a dope comprising 33% wt 18B powder and formic acid was created using a FlackTek planetary mixer.

FIG. 1 illustrates the components and processes used to generate the high-friction fibers (“spinline”). The dope was extruded using extrusion device 10 through a line 12 to a 50-orifice spinneret 14 within a coagulation bath 16. The 50-orifice spinneret 14 was used to produce a 50-fiber tow of fibers, where each orifice was approximately 127 μm in diameter. The dope was extruded at a rate of 2 ml/minute into the coagulation bath 16 comprising 87% by volume ethanol and 13% by volume formic acid. The coagulation bath 16 was 2.97 meters in length and maintained at room temperature (˜22° C.). The fibers were passed through the coagulation bath 16 and taken up by a first three-roll haul off machine 18 at a rate of 12 meters/minute. Based on the extrusion rate and the takeup rate from the first three-roll haul off machine 18, the fibers were subject to a draw ratio of 3.75× while in the coagulation bath 16.

The fibers were then passed through an ethanol bath 20 that was 2.99 meters in length comprising 100% ethanol maintained at room temperature (˜22° C.). The fibers were taken up by a second three-roll haul off machine 22 at a rate of 13.2 meters/minute. As there was minimal difference in the takeup rates of the first three-roll haul off machine 18 and the second three-roll haul off machine 22, the draw ratio that the fibers were subject to in the ethanol bath was approximately 1.1×.

The fibers were then passed through a third bath 24 that was 2.99 meters in length comprising 91.9% by volume ethanol and 8.1% by volume double-ionized water. The third bath 24 was maintained at room temperature (˜22° C.). The fibers were taken up by a third three-roll haul off 28 at a rate of 19.2 meters/minute to provide a draw ratio of 1.45× in the third bath 24 based on the difference in takeup rate from the second three-roll haul off machine 22.

As the fibers exited the third bath 24 and before entering the third three roll haul off 28, they were passed over series of air flows 26 comprising an air blade (not displayed) and a turbulent air flow (not displayed). The fibers were first passed over the air blade set at a pressure ranging from 15 to 35 psi. The fibers were then subject to a turbulent air flow set at a pressure ranging from 15 to 35 psi.

The fibers were then passed through a dryer 30 set at a temperature of 15° C. The total residence time in the dryer 30 was approximately 45 seconds. The fibers were then taken up by a first five-roll haul off 32 at a rate of 20.4 meters/minute. The total residence time in the dryer 30 was 45 seconds and the draw ratio that the fibers were subject to in the dryer was 1.06×.

After leaving the dryer 30, the fibers were passed through a first device 34 comprising a uniform hot surface heated to a temperature of 160° C. The fiber was passed over a 40 cm length of the hot surface and was taken up by a second five-roll haul off 36 at a rate of 44.4 meters for minute to provide a draw ratio of 2.2× as the fibers were passed over the uniform hot surface.

The fibers were then passed through an oven 38 that provided an ambient heat temperature of 150° C. The humidity within the oven 38 was not controlled and the oven 38 was 1.5 meters in length. The fibers were taken up by a third five-roll haul off 40 at a rate of 44.4 meters/minute to provide a draw ratio of 1× for the fiber as it was passed through the oven 38. The high-friction fiber was then collected on spools 42 for production into staple yarn and further analysis.

### EXAMPLE 5

### Coefficient of Friction for Low and High-Friction Fibers

The coefficient of friction for the low-friction fibers, high-friction fibers and polyester was measured according to the ASTM 3808 standard. Specifically, the input and output friction was measured at a rate of 3.0 meters/minute and at a 168-degree wrap angle. For each sample, a length of at least 6 meters was tested at intervals of 1cm to produce a mean coefficient of friction.

For each set of samples, the median mean coefficient of friction was calculated and was tabulated for the various sample below along with the standard deviation of the mean coefficient of friction, the average of the longitudinal coefficient of variation for the mean coefficient of friction and the number of samples used to produce the mean coefficient of friction.

TABLE 5
Mean Coefficient of Friction for High-Friction Fiber,
Low-Friction Fiber and Polyester

| | | Standard Deviation | Average of | |
|---|---|---|---|---|
| | Mean | of the Mean | Longitudinal | Number |
| | Coefficient | Coefficient of | Coefficient of | of |
| | of Friction | Friction | Variation % | Samples |
| Low Friction | 0.57 | 0.068 | 11.32% | 9 |
| Fiber | | | | |
| High Friction | 0.89 | 0.11 | 30.25% | 229 |
| Fiber | | | | |
| Polyester | 0.38 | n/a | 8.95% | 1 |

### EXAMPLE 6

### Scanning Electron Microscopy of Low and High Friction Fibers

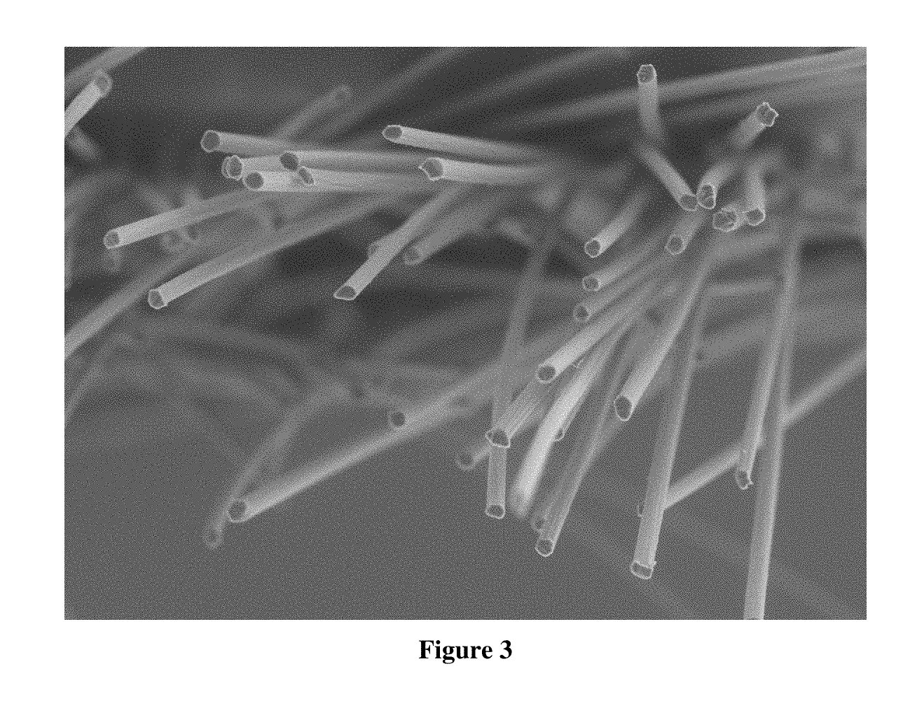

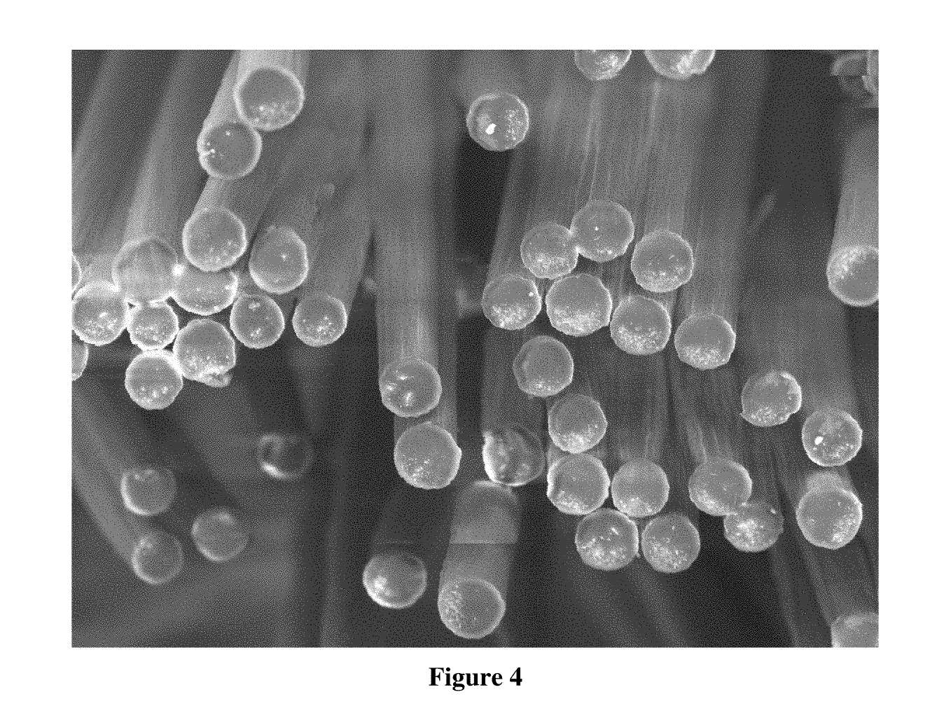

FIG. 2, showing an image of a low friction fiber and FIG. 3, showing an image of a high friction fiber, each produced by the methods described herein, were each generated using scanning electron microscopy at a 200× magnification. As can be observed, the high-friction fibers had ridges on its outside surface whereas the low-friction fibers were smooth. It was observed that the high-friction fibers exhibited less fusion than the low-friction fibers. The low-friction fibers were substantially unfused and whereas the high-frinction fibers exhibited fusion. FIG. 4 provides a higher magnification (600×) image of the high-friction fibers that clearly shows the ridged structures.

### EXAMPLE 10

### Fiberwebs formed from High-Friction Fibers and Wool

Various amounts of high-friction fibers were cut into staple and carded with wool to generate fiberwebs which was then pin-drafted to produce a roving that was spun to make yarn. Loss at various stages of the process was assessed is included below as Table 6.

The high-friction fibers were cut into staple fibers ranging from 2-25 inches in length. Wool top was treated with a pre-conditioning spray comprising a mix of 99% water and 1% syntholube and passed through a Morley brand fiber opening machine. The wool top was then combined with the high-friction fibers and passed through the Morley brand fiber opening machine a second time to blend the fibers. The overall percentages by weight of wool and high-friction fiber were approximately 60% and 40%, respectively.

After opening, the high-friction fibers and the wool top were passed through a carding machine at a rate of 2.52 lbs/hour and a rate of 4586 revolutions per lb. The carded high-friction fibers and wool top was then pin-drafted at varying speeds. The pin-drafted high-friction fibers and wool top was then spun into yarn at varying speeds.

The amount of waste throughout the production process was quantified in order to assess the goodness of the high-friction fiber at forming fiberwebs. Exemplary results on waste from processing from various lots of fiber are included in Table 6 below. As is shown in Table 6, waste at the carding stage ranged from 2.55-4.61% with the overall waste throughout the process ranging from 12.03-13.09%

TABLE 6
Waste generated during processing of high-friction fibers and wool top.

| | | | | | Pin | | | | |
|---|---|---|---|---|---|---|---|---|---|
| | High | | Card | Card | Drafting/ | Pin | | | |
| | Friction | Wool | Pass | Pass | Spin | Drafting/ | | Total | |
| | Staple | Top | Loss | Loss | Loss | Spin Loss | Final | Loss | Total |
| | Fiber | (lbs) | (lbs) | (%) | (lbs) | (%) | Weight | (lbs) | Loss % |
| Lot | 1.88 | 2.82 | 0.12 | 2.55% | 0.21 | 4.58% | 4.09 | 0.62 | 13.09% |
| 1 | | | | | | | | | |
| Lot | 1.88 | 2.82 | 0.19 | 4.04% | 0.16 | 3.54% | 4.06 | 0.30 | 13.73% |
| 2 | | | | | | | | | |
| Lot | 0.94 | 1.41 | 0.11 | 4.68% | 0.1 | 4.46% | 2.07 | 0.28 | 12.03% |
| 3 | | | | | | | | | |
| Lot | 1.65 | 2.47 | 0.19 | 4.61% | 0.11 | 2.80% | 3.61 | 0.50 | 12.33% |
| 4 | | | | | | | | | |

1. A plurality of fibers comprising recombinant spider silk polypeptide, wherein the plurality of fibers are generated by:
dissolving a powder comprising a recombinant spider silk polypeptide into a solvent to generate a spin dope;
extruding the spin dope into a coagulation bath to form a plurality of precursor fibers; and
subjecting the plurality of precursor fibers to a turbulent air source, thereby generating the plurality of fibers comprising recombinant spider silk polypeptide,
wherein one or more of the plurality of fibers has a mean coefficient of friction that is greater than 0.60.

2. The plurality of fibers of claim 1, wherein the plurality of fibers is an unfused plurality of fibers.

3. The plurality of fibers of claim 1, wherein the plurality of fibers forms a sufficient mechanical interaction with wool to form a web of fibers.

4. The plurality of fibers of claim 1, wherein one or more of the plurality of fibers has a mean coefficient of friction that is greater than 0.70.

5. The plurality of fibers of claim 1, wherein one or more of the plurality of fibers has a mean coefficient of friction that is greater than 0.80.

6. The plurality of fibers of claim 1, wherein one or more of the plurality of fibers has a mean coefficient of friction that is greater than 0.85.

7. The plurality of fibers of claim 1, wherein the powder comprising the recombinant spider silk polypeptide is comprised of at least 55% recombinant spider silk polypeptide by weight.

8. The plurality of fibers of claim 1, wherein the mean coefficient of friction is measured using the ASTM 3808 standard.

9. The plurality of fibers of claim 1, wherein the mean coefficient of friction is measured at intervals of 1 cm along a length of at least 6 meters.

10. The plurality of fibers of claim 1, wherein the fiber is produced by a continuous wet-spinning process.

11. The plurality of fibers of claim 1, wherein the plurality of fibers comprises more than 30 fibers.

12. The plurality of fibers of claim 1, wherein the plurality of fibers comprises more than 50 fibers.

13. The plurality of fibers of claim 1, wherein the plurality of fibers produces a loss in weight of less than 4.68% when carded with a blend of 60% wool by weight.

14. The plurality of fibers of claim 1, wherein the plurality of fibers produces an overall loss in weight of less than 13% when processed into yarn with a blend of 60% wool by weight.

15. A staple yarn comprising wool and the plurality of fibers of claim 1 divided into discrete lengths.

16. A knitted garment comprising the staple yarn of claim 15.

17. A recombinant fiber comprising a recombinant polypeptide, wherein said fiber comprises a mean coefficient of friction of greater than 0.60.

18. The recombinant fiber of claim 17, wherein said fiber comprises a mean coefficient of friction of greater than 0.70, greater than 0.80, or greater than 0.85.

19. The recombinant fiber of claim 17, wherein the mean coefficient of friction is measured using the ASTM 3808 standard.

20. The recombinant fiber of claim 17, wherein the mean coefficient of friction is measured at intervals of 1 cm along a length of at least 6 meters.

21. The recombinant fiber of claim 17, wherein said recombinant polypeptide is a recombinant spider silk polypeptide.

22. The recombinant fiber of claim 17, wherein said recombinant polypeptide is a silk-like polypeptide.

23. The recombinant fiber of claim 17, wherein said recombinant polypeptide comprises repeat units, wherein
each repeat unit has at least 95% sequence identity to a sequence that comprises from 2 to 20 quasi-repeat units,
each quasi-repeat unit having a composition comprising {GGY-[GPG-X1]n1-GPS-(A)n2}(SEQ ID NO: 34), wherein for each quasi-repeat unit:
X1 is independently selected from the group consisting of SGGQQ (SEQ ID NO: 35), GAGQQ (SEQ ID NO: 36), GQGPY (SEQ ID NO: 37), AGQQ (SEQ ID NO: 38), and SQ; and
n1 is from 4 to 8, and n2 is from 6 to 10.

24. The recombinant fiber of claim 23, wherein n1 is from 4 to 5 for at least half of the quasi-repeat units.

25. The recombinant fiber of claim 23, wherein n2 is from 5 to 8 for at least half of the quasi-repeat units.

26. The recombinant fiber of claim 23, wherein each quasi-repeat unit has at least 95% sequence identity to a MaSp2 dragline silk protein subsequence.

27. The recombinant fiber of claim 23, wherein the repeat unit comprises SEQ ID NO: 2.

28. The recombinant fiber of claim 17, wherein said recombinant polypeptide comprises SEQ ID NO: 1

29. A plurality of recombinant fibers of claim 17.

30. The plurality of recombinant fibers of claim 29, wherein the plurality of fibers is an unfused plurality of fibers.

31. The plurality of recombinant fibers of claim 29, wherein the plurality of fibers forms a sufficient mechanical interaction with wool to form a web of fibers.

32. The plurality of recombinant fibers of claim 29, wherein the plurality of fibers comprises at least 30 fibers or at least 50 fibers.

33. A staple yarn comprising wool and the plurality of fibers of claim 29 divided into discrete lengths.

34. A knitted garment comprising the staple yarn of claim 33.

35. A knitted garment comprising a staple yarn, wherein said staple yarn comprises wool and a plurality of recombinant fibers divided into discrete lengths, wherein said plurality of recombinant fibers comprise silk-like polypeptides, and wherein said plurality of recombinant fibers comprises a mean coefficient of friction of greater than 0.60.

36. The knitted garment of claim 35, wherein said plurality of recombinant fibers are generated by:
dissolving a powder comprising a recombinant spider silk polypeptide into a solvent to generate a spin dope;
extruding the spin dope into a coagulation bath to form a plurality of precursor fibers; and
subjecting the plurality of precursor fibers to a turbulent air source, thereby generating the plurality of recombinant fibers comprising the recombinant spider silk polypeptide.

37. A method of generating a fiber comprising recombinant spider silk polypeptide, comprising:
dissolving a powder comprising a recombinant spider silk polypeptide into a solvent to generate a spin dope;
extruding the spin dope into a coagulation bath to form a precursor fiber; and
subjecting the precursor fiber to a turbulent air source, thereby generating the fiber comprising recombinant spider silk polypeptide,
wherein the fiber comprising recombinant spider silk polypeptide has a mean coefficient of friction that is greater than 0.60.
